SEQUENCE LISTING (24 sequences)

SEQ ID NO: 1 - DNA, 4596 bp, synthetic construct
  misc_feature 1..4596: note = A2M DNA sequence
  source 1..4596: mol_type = other DNA
gagatgggattgttaatgagtagggtacaatacaggctagaatccatctggtctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgtcgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggggtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtcagtgtgcagctggaagcctctcccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttgaagaccacaaggctgaaaagtgctttgctggagtcctgttctcagagctccacagaagacacgtgtttttgtatctttaaagacttgatgaata

SEQ ID NO: 2 - protein, 1474 aa, synthetic construct
  REGION 1..1474: note = A2M amino acid sequence
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 3 - DNA, 20454 bp, synthetic construct
  misc_feature 1..20454: note = CKM DNA sequence
  source 1..20454: mol_type = other DNA
ctaatttttgtacttttagtagagatggggtttcaccatgttggccaggctggtcttgaactcctggcctcaagtgatccacccgcctccgcctctgcctcccaaagtgctgggattacgggcctgagccactgtgcccggcccatctaacattttcactgtcaatcacaatgggattaaaactcctcccacagcccctagggcaccaccatgggtctgctcctgtctcccctccaacctcatcttcttcctcccactctctccttggccccatctgctccagtcccctggcctccttcctgtctgtcctcagatgtgcccagccattctcacctcagcgcctttgcacctgctgttccccccagagccgcacatggctggctccctgttctccttcaggtctctgctcagatgtcatcttcccaaagaggcctgcctcgacctcccctgctgctgtgccgtcccctcatctgtgaccctcttgcactatcacctccaggacggcgggggttttgtgttttgttgtagcctcaggaagtgcctgatagatccctgtttcgagaccagttccatttggttttctgggcctcagtttccgtaaccgtgaaggagaccctcggcaatctgagcttgctgggaaagggctgggccccatgtaaatatttctaaagcacccctctcccctcccccctcagatcaggagtctgagggagaggcacagaggctccctttctctaagccagtcctcacctgcctaagaagatgtgaaggagacccaggagaccctgggatagggaggaactcagagggaagggacattcttttcttcgtcgcaatcctgggagctccctggaggaggagacccgatcagcctgcaatcctggcgcgtcccaggaggagaaagcggcttcctctatactgtactctcctccacagaacccccctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagacagccccccttcagcccagcccagccaggtactgcacggggcgggaatctgggtgggggccagagtaggggatttctgtgggtgctagaggcttggcttgggaaagggtctgtgtgtcaccccttgctccaccaacatcctcctatacaaaggcaggtcggtgcgtgggaaggttgacccttgtgtgtctgggaggcccctccatctgtgaggctgcctgaaccccccactgggacctgtgatttctgcggcacaagtcttgggttatggggagggatccgagatgaaaggggtcctgacagctgccaactgtcaccccctgcaaggggctgttgagccaggcctgtgttcattacagtttgtcgccaacactggcgccacaagccgtgggaggtgtgtctgtcgcctagcagagtctgcaagagcaggtgtcgcaggctttctttgcgtttgctactgtgtgggtccctgtgaccttgtatctgacttagtagcggacacatgagtgtcctgggcatgtccttgagtttggaagtgtgactgtgtgacttagtttacagtgtgatctggggagtctgcggtggtgggggtgtgtggctctggggctttctgcttatgtctcttgtcgggggggtcaccaccctgcctcagtgccctctcctggggtgtccctggcagggattaggggggcatcctctgaagggagggggtctgggctgccatcccaggatgtccttgagccccgaggcctggacccgtgtcctcccagccaccctggcccacctgtcttgggcctgctgacttggtgcaagccgagccccggaatgtgggggtggggcggggcagcagctgggggtgggatggcaggcagttgccagattctaaaaataaccaccctggacagcagcacccaaggtctttggcgggtccctgttcctcttccctggcttcagctcaccgtggtcccaatctccctcccacaatcccacctgcttcccatccccgtctcccaccgctgctggaggccccttctcctttctgggtcccttctcctttctgggtccagtgaataaaataataataacagtaggacctggcgcagtggctcacacgcataatcccagcatcttgggaggccgatgctggaggatcacttgaggccaggagtcccagaccagcctgggcaacatagtgagaccccatctctgcaaaaaagtagtaataacaaataacaataatagcagccaccagggttccaggctccaactccatgccagggcctgtgctcagccaacctcatggccaagctcagaggatcctggcaatcctacggggcaggctcagttcacaggagacagaggtccagagaggcctagtgactggcctggggtcacacagcagggaaatggcataggtgggatttgaacctgcctctgacgacccagaggctgagcttttcttcccttctgagccctgtgggagaactgtcagcaggcagaggtgggggcagctacccagccctcccccgacccagccctttgtctttggattgataatccaagaacctgctcacatactcccagcagcaccctcacctccaccttctcaccagctcctcgcacgaacctttaaaataacaacaacaggccaggtgccgtgactcatgcctgtaatctcagcactttgggaggcagaggtgggcggatcacttgagttcaggagttcaagaccagcctggccaacatggcgaaaccccaactctactaaaaataccaaaaaaaaaaatagccaggtgtggcacacgcctgtagttgcagctactcaggaggctgaagcaggatagttgcttgaacctgggaggtggaggttgcagtgaactgagattatgccactgcactctagcctgggcgacagagcgagactctgtctcaaacataataataataataataataatagcagctaagtgaaataatgcttattgtgtaccaggcactgttccgagagctacacctgtattcacacatttaatcttttcaacactcccatgcggcaagtgctgttaggatcctcattttctagatgagtaaattgaggcacagaggttaagcaacttgcccctggccacacagctggttgtaggtagcagagctgggaatcaaaccctgggctgattccagaacctacactcttacccacacaaaatatttttccagcctccgggaggggcccgttttccacctggggaaattgagtttccagcaagaatgactagtccaggctcacccaactgggatacggcagagccaggcccttgatctaagactccggatgccttcctgctgagacacaggggctcagagagaggatttctcttgctccaagttacacagcctgcctgaccccaacgccttgcccagttctcttgctgctgtctgcaagacgcgagctaggcggggcacagtggctcacgcctgtaatcccagcactttgggaggccgaggcgggtggatcacttgaggccaggagttcgagaccagcctggccaaccagtagagatggtgaaaccccatctctaataaaaatacaaaaaaattagccaggcatggtggtgtgcgcctgtaatcccagctactcaggaggctgagacaggagaatctcttgaacctgggaggtggaggctgcagtgagccgaggtctcgccactgcactccaacctgggtaacagagtgagactccatctcaaaaaaaagaagaaaaggccgggtgcagtggctcacgcctgtaatcccagcactttgggaggccgaggcgggcggatcacaaggtcaggagatcaagactatcctggctaacatggtgaaaccccgtctctactaaaaatacaaaaaatgagccggcgtggtggcgggcgcctgtattcccagctacttgggaggctgaggcgggagaatggtgtgaacccaggaggtggagcttgcagtgagccgagatcgtgccactgcactccagcctgggtgacagagcgagactccgtctcaaaaaaaaaaaaagaagaagaagaagaaaaaagatgccagctaatcttcaatcttcccctcctgccctgcccatcaggtctcctacaccgccaccatgccattcggtaacacccacaacaagttcaagctgaattacaagcctgaggaggagtaccccgacctcagcaaacataacaaccacatggccaaggtactgacccttgaactctacaagaagctgcgggacaaggagactccatctggcttcactgtagacgatgtcatccagacaggagtggacaacccaggtgagcctccccagtggagcactgaaggggctacatgggggctctggaggctgccggccatgccccaatcccaccacccttgaagggggggggggcctcagttttctcatctgtaaaatggggctgttgtgggaatcaatgcattaatatacataaaggcttgggacagtgcgaggtactcagcaaaccacgcaataaacatgcacgagaataatgtgagatccccaaggaggtcggttcacctcctgaacctcagtctcctcatctgaaaaatgggcgcccacgttgtgaaaacacaagtcaatggctgtgcagtgcttagagtggcacctggcaccaagtgagtgctcaagctctgacaggggggccaggtgcagtggctcatgcctgtaatcccagcactttgggaggcccaggtgggaggatcacttgaggccaggagtttgagaccagcctgagcaacatagcaagaccctgtctctacaaaaaatacaaaaaaattagccaagtgtggtgatgagggcttgtggtccaagctactcaggaggctgcggtgggaggatcgcttgagcctgggaggtgcaggttgcagtaagccgagatcgcgccactgtactccagccacagagtgagaccctgtctcaaaaaaaaaaaaaaagaatctgacaggggatgttattaatgttatggttgtttctcaaaggacattgcaaggcgaagagagctgggagtggaggggaggctctagtgcccctcacatccagctcactgtgtgatgccggggagaagcccctctctcctggcccctgtttttccactgggaaacaagagggtctttcaagtctcttaccctcctgaccctctgtggtacggtggtagcctgagatcctaacatttcaggctaagatgtcatgactttgggtgctaatgtttccgaggtgctgatgcttttagcaccatgtaccctctgaagctcccctttctcctttgaccccgcccccagcactctttcttctctcccaccttgacccctcccagccaccttgacctctcccagccttccctcccagttcccaccagtcctcagtgttctctctttttttttttttcctttggagacagagtcttgctctgtcacccaggctggagtgcagtggtgtgatcttggctcactgcaacctccactcccttggttcaagcaattctcatgcctcagcctcctgagtagctgggattacaggtgtgtgccccacacccagctaatttttgtatttttagcagagatggggtttcaccctgttggccaggctggtctcacactcctgacctcaagagattcacccgcctcaccctcccaaagtgctgggattgcaggcatgagccactgtgcccaacccttatgattctcaattcccacccagaggtattgaatgtacccgcccccatcccgacacacatgtccacacaggctccactgagcccacaagcccacgccctgtttgctggggacaagcagctctgacctcacccccacccctccaggtcaccccttcatcatgaccgtgggctgcgtggctggtgatgaggagtcctacgaagttttcaaggaactctttgaccccatcatctcggatcgccacgggggctacaaacccactgacaagcacaagactgacctcaaccatgaaaacctcaaggtcggtgtctgcgcaggaggggagggccgaggggtgaggaggagagagatcacaggagaaatggggggagacagagaaatacagagacagagagagacacagaggtgtggagaagcagagagatggaaagagagagggacattgggaggctgaggcgagcgtatcacttgaggtcaggagttcaagaccagcctggccaacatggcaaaactctgtctttaaaaaaaaaaaaaagggctgggcacggcggtggctcacgcctgtaatcccagcactttgggaggccaaggcgggcagatcaccttagattgagagtttgagaccagcctgaccaacatggtgaaaccccgtctctactaaaaatacagaattagctgggtgtggtggcacatgcctgtaatcccagctacgtgggaggctgaggcaggagaatcgcttgaacctggaaggcagaggttgtggtgagcagagatcgtgccattgcactccagcctgggcaacaagggcgaaactcgtctcaaaaaataaataaataaataaataaaataaattagctgggcatggtggcgcgtgcctgtaatcccagctactcaggaggtggaggcaggaaaatctcttgaacccaggagatggaggctgcagtgagctgagatcatgccactgcactccagcctgggtgacagagcaagattctatctcagaaaataaaaaaaggaaagagagagggacagagagacacagagacagccaaggagacacagacaaccaaggagacagacagacaaaggagagggagagggagaagaaaggcacaaaagcatgtagaaaaggaagaggcatagccgggcacagtggctcacgcttgtaatcccaacactttgggaggctgaggtgggcggatcataaggtcaggagttcgagaccagcctgaccaacacggtgaaacccagtctctattaaaaatacaaaaattagctaggcatgctagtgttcacctataatcctagctattcagcctgggtgacagagcgagactctgcctaaaaaaaaaagggtggggggagaggcagagacacagagggagacagaagggagcatcagagaaggcaggagagacagggagagagagacacacgcagttgcagtcagacacagacaaggagagtgggagaaagggagacaggaagagaaacagaaatgggcagaaaggctgggtgcagtggctatacctgtaatcccagcacttatggaggctgaggtgtgaggatcacgtgaagccaggagagttcacgaccagcttgggcaacatagcaagaacccgtctctgcccaaaaaaaagaaaaaaaaaagcctaaaaatttagctaggcatggtggctcatgcctgtaatcccatcacgttgggaggctgaggcaggaggactgtttgagcccaggagtttgagaccagcctgggcaacatagtgacaccctgtctctattttttttttttttttgagacagaatctcactctgtcacccaggttggagtgcagtggctcgatctcagctcacggaaacccctgatccccaggttcaagtgattctcgtgcctcagcctcctgagtagctgggattacaggcgcgcaccaccacgcccagctaatttttgtatttttagtagaaatggggtttcaccatgttggccaagctggtttcgaactcctgacctcaggtgattggctcacctcggcttcccaaagtgttgggattacaggtgtgagccaccgcgcccagcccctgtctctatttataaaaaatttattttaagttagccgaacatggtggcatatacctacctgttagtcccagctacttgggaggctgaggcaggggaattgcttgagcccaggagtttgaggttgcagtgagctatgatggcaccactgcactccagcctgggcgacagagagagaccctgtctcgaagaaaaaaaaggaaaagaaatatacgaagatggggagagagaggtataaagaaaaacagagataggagaggggagagagacagagaagggagagacagagcccaggtatgggtggcggggagaggctccacacacccaatcagtgaggatgcagaaggcaaaggaagcaacagggagactgaggggagcatctgggggagacctgggcttgctggagagggcggaggatctgcctggtcctgatctcccacccgctctccagggtggagacgacctggaccctaactacgtgctcagcagccgcgtccgcactggccgcagcatcaagggctacacgttgcccccacactgctcccgtggcgagcgccgggcggtggagaagctctctgtggaaggtgagcgcccctaccccgtccactgctggggtcacccagccctggctctgttcctccatgggcaggtcctcgggtctctttgggcccccgcttttcaaagtaggggaagagcttggtaaatcgtgggggcttggggaatggcgcgggctgtcagcattacctgctgtgttggagattcagggatgctgtccagacccgacggcccctgagaagacctacgcagacacctggctgcccccagcgtcctgccatccgttcatccagcacacatttccaggggttcttgccctgtgtggggagataggggtccctcactgttctcacagagtgttcagctggtggggagacaagaggaaatacagaaacccacagatgaacaagataattcaaaagtgccatgaagccaagacatgggtgtggtggagaatgaccgaggggagccacctcagataggaggagttgaggccgggcacagtggctcccacagcagtgcgggaggcctaggagggagggtcgcttgagcccaggagttcaagaccagactgggcaatacagcaagaccctgtctctacaaaaaagacaaaaatgagccaggagtggtggcacacacctgtcatcccagctactcaggagactgaggtgggaggatcacttgagcccgggaggttgaggctgcagtgaatcgtgattgtgccactgcactccagccagggtgacagagtgagaacctgcctcaaaaacaaataaaaagaggccaggcacagtggctcacaccgtaatcccagcattttgggaagctgaggcaggtggatcatttgaggtcaggagtttgagaccagcctggccaacatggtaaaaccccatctctactaaaaatacaaaaatcagctgggtctggtggcacacacctgtaatcccagcgacttgggagactgaggcaggagaatcgcttgaacctgggaggcagaggttgcagtgagctgagatcacgccactgcactccagcctgggggacagagtgtgactccatctcaaaaaaaaaagttatgtttatcctatactgtagtctgttaagtgcacaatagcattatgtctagaaaaaaaaaagtacataccttaattttaaaatcctgctaaaaatgttgtgatcatttgaggactcagcaagttggaacctttctgttgtgtagggccttgccttgatgttgatgggctgctgactgatcagggcggttgtcgttcaaggttgagatggctgcggcaattccttccttccttccttccttcctttcctttcttttcttcctcccttccttcccttccttccctccctcccttccttcccttcccttcccttttcttctccgtccttccttccttccttctttcctccctcccttcctcccttccttccttccttctctttctctctctcttttcaagacagggtcttgttctgttgcccagctggaatgcagtggtgcaatcatgacttaccataacctccaactcctgggcttaagtgatcctcccatgttggtctcccaaagtgctggcattacaggtgtgagccacagtgcctggccagcagtaatagtttgaaaagaatctcttttttgagatggagccacactcttgtcgcccaggctggagtgcagtggtgcaatcttggcttactgttacctccacctcgtggttcaagcaattctcgtgtctcagccacccaagtagctgagatcaccggcatgcgccaccacgcctggctaatttttgtatttttagtagaatcggggtttcgccatcttggccaggctggtctcaaactcctgatctcaggtgatctgcctgcctcagcctcccaaaccactaggattacaggcgtgagccaccgcacctggcctatgaagctttttatttaaagtgaaaaatgttgactcttcctttcacttgaacacctcgaggccattgtaaggttatgaactgaattggcctgatttcaaaatcatgtgtctcagggaatacggaggctcaaggagatggggagagatgagggaatggctggtcagtggggcagtccgagcacacacatttatccattaagttcacccttttatatgggcaaggtcggtggcaccccccaaaattaaaacagtaacatcagagatcactgatcacggatcgccatgacagatatagtaataatgagaaagtctgcagtactgtgagaattacccacatgtgacacagagacacgaagtggcacatgctgtaggaaaaatggctccaatagacctgctggacaaagagttgccacaaacctccaatttgtaaaaaacacagtatctgcaaaatgcaataaaacgaggtatgtctgtatattatatgaatatgtataagatatataaatattatagatttattgtacatatgcacaaacacaaattcattatacacatattttatacatgaatatacaaataaacacacaattttttttcttttttttttttttagacagaatctcactctgtcacccaggctggagtgcagtggcaaaatctcagctcactgcaacctccacctcttgggttcaagtgtttctcgtgcctcagcctcttgagtagctgggattacaggcgtgtgccaccatggccgactaatttttgtatttttagtggagacggggtttcaccatgttggccaggctggtctcaaaatcctgacctcaagtgatctgcctgccttggcctcccaaagtgctgggattataggcatgagccactgcgcccagcctacaatttttttttttttaaagattggggtcttgggccaggcgcccaagtggagaaaccccgtctctactaaaaatacaaaattagccgggcatggtggcgcatgcctgtaatcccaggtacttgagaggctgaggcaagagattcgcttgaacccgggagacggaggtggcagtgagccaagatcgcgccattgcgctccagcctgggcaacaagagcaaaactccgtctcataaaaaaaaaaaaaaaagatgggggtcttgctatgttacccagactggactcgaactcctgggcttaagtgatcctcctgcctcagcctccccagtatctggactacaggctcgtgccagtgcacctggctaccattctactttataataatttttactaatggcctctcaaagtcctgagacttcagccatctgtgttgccagccattaggagcaggcaccagcacactgtggcctgcaggtggattggatgtgagggtccctcaggggaagaggtgggtctgttttccagattgagcacatggggtggccattcctgggggcctggacagatggcaggaacagattttggggagccgtcacaagtgtgggtttagacacagggttcaggtgcctttaggacatccagggggccaaggcccaggcccgttaggagcagcagcagggggaaccctgggcctccacaccctggttgaggtgggacaggctgctgaccactccctcgtgtgtccccatagctctcaacagcctgacgggcgagttcaaagggaagtactaccctctgaagagcatgacggagaaggagcagcagcagctcatcgatgaccacttcctgttcgacaagcccgtgtccccgctgctgctggcctcaggcatggcccgcgactggcccgacgcccgtggcatctggtgaggcccctccccgccccctggcttcctcttcccccagcctcctccagtcctgcaacggccctcctcactcctccaccaaaaccaaggccatatacataattatgatcttctaaatgccacggaaaatgatcttgccctccgcccctgcccatcctgtggtctgtgtgtcagattttgggatatctggaccggagaagtggtagttaaaaacttaagacttggcggggcacggtggctcatgcctgtaatccctgcactttgcgaggccaaggcggggggatcacttgaggtcaggagtttgagaccagcctggccaacacggtgtctactaaaaatgacaaaaatttagctgggcgtagtggcaggcgcctgtagtcccagctacttgggaagatgatgcaggagaatcgcttgaacccaggtggcggaggttgcagtgagccgagatcgtgccactgcactccagcctgggcgacagagccagactgtctcaaaaaaaaacaaacaaaaaagaaagaacttagacttggccgggcacagtggctcacacctgtaatcccagcactttgggaggctgcagcaggagggtcgcttgtgcccaggagttctagaccagcctgggcaacatagggagaccacgtcgctacaaaaattaaaaaaaaaaaaagccaggcgcggtggctcacgcctgtaatcccagcactttgggaggccaaggtgggcggatcacgaggtcaggagattgacaccatcctggctaacacggtgaaaccccgtctctactaaaactacaaaaaaaaaaaaaaaattagcccggcgtcgtggcaggcgcctgtagtcccagctacttgggaggctgaggcaggagaatggcctgaacccaagaggcggagcttgcagtgagctgagatcgtgccactgcactccagcctgggcgacaggagactccgtctcaaaaaaaaaaaaaaaaaaaaaaaaaaaaaaaagccaggcgtgatggcgtatacctgtggtcccagctactcgagaggctgaggatcagctgggcccagaggattgaggttactgtgaatcatgaccgaggtactgcactccagctacctgtctcaaaaataagaactcagacttgaaccgtactccctaggttcaaatcccagctctaccatttactagatttactagctttgtgaccttgggcaacttattttatttttctctaccccagtttcctagctataggatagggatcacaataatttgtagtttctggtgttcttgtgaggattaaatgagttaatttatgtaaagtattaaaagcagtgcctgggccaggcgcggtggctcacgcctgtaatcccagcactttgggaggccaaggcgggcggatcacgaggtcaggagatcgagaccatcctggctaacatggtgaaaccccgtctctactaaaaatacaaaaaattagctgggcttggtggcaggcgcctgtagtcctacttactcgtgaggctgaggcaggagaatggcgtgaacccgggaggcggagcttgaagtgagccaagatcgcatcactgcactccagcctgggcgacagaccaagactctgtctcaaaaaaaaaaaaaaaaaaaaaagcagtgcctggtatacactaagtgttcagtacatcttggttattcgtcttatctttattataaagtattattgctagacttgttttgactctttgaatccctgattcagatagtcaaagatttagagaacctaggtttcaatcgatgaagggagcctgggtgctaagatatacagatgggatgattctaagatttcaagattttaaatatttttaggttttaagattccactcatttatttatttatttattttgagatggactctcactctgaggcccaggctggagtgcagtgggttgatctcgactcactgaaacctccatctcccgggttcaaacgatcctctcaccctccgcctccttagtagctaggacgacaggcatgtgccactgtgcccagctagttttcgtatttttggtagagacagcgttctgccatgttgctcaggctggtctcgaactcttcaccttaagtgatccgcccacctcagcctcccaaagtgctgggattacaagcgtgagccaccatgcctgccctcaggtttttttttttttttttttgagacagggtcttgctctgttgcccaggctggagtgcagtggtgggatcatagctcgctgcagcctggaccttccaaggtcaagtgatcctcccgcctcagcctccccagtggcttggactacaggcatgtgtcccaccatgcctgctaattttttaaattttgtagatatgtgggtcttgccatgttgcccagggtagtcttgaactcctgggctcaagcaatcctccggccttggcctcccaaagtgctgggattacaggcatgagccaccacacccggccatttccggatcttaatattttatgttcttttttttttttttttttgagatggagtctcgctctgttgcccaggctggagtgcagtggcgcgatctcgactcactgcaagctccacctcccgggttctcgccattctcttgcctcagcctcctgagtagctgggactacagatgcccgccaccatgcccagatttttttttgtattttttagtagagacagggtttcgacgtgttagccaggatggtgtcgatctcctgaccttgtgatccgcccgcctcggcctcccaaagtgctgggattacaggcgtgagccaccgcacccagccttaatattttatgttctaagatcctgcttctgagatgctaggattgggagctaacacactacgatgggaagattttctggtcctgaaaccagatgtaggatttccaagtgttatcagttctgatcccaagcttctgtttgtcaaacatcctgagatcccaaggatctataataatactgttcaaagacttaagtccctaaaagtccttgatcccaggattcagcgttttgcagatcctgtgacattagagtctacaggtttaagcttctgcgttttgaggaggcagatgccacccacctcccacccctcaggccctcttcccccatcggtgggcagatgttttgggggagggctgggaatggggttcccacggggctgacaccttggcctcttgctgcggcaccttactctaggcacaatgacaacaagagcttcctggtgtgggtgaacgaggaggatcacctccgggtcatctccatggagaaggggggcaacatgaaggaggttttccgccgcttctgcgtagggctgcagaaggtgggtgtctgcccttagctgctgacttctgactccaccttgaacgccccacctgctttccgaggccccgccccttcccctatactaagccctgccccacccattttccagggccctgccccccggacctggctccaccccatgccaaaccccgccccccaaaactagccccacccgcttttcagggccccgcccctggacccagctccaccctatgcccaagccccgcccgtaaggatccgccttcaggccgggagcggtggctcacgcctgtaatcccagcactttgggaggctgaggagggtcaggagttcaagaccagcctggccaacatggtgaaaccccgtctctatactaaaaaattaataaataaataaattatccaggcgtagtggcgcgcgcctgtagtcccagctactcgggaggctgaggcaggagaatcgcttgaacctgggaggcagaggttgcagtgagacgagatcacgccattgcactccagcctgggtgacacagagagactctggaaaaaaaaaaaaaaaaaacccgccttcaaagcgttcttagtccccgcccctcaaaatcctgccttggtccccattgtctagccagcaccctcggaatctcctacctccctgttcccctcagaatcccacctccaccacgttcccacgccctgtccctccggaccctgtccatgttgcgggtgcatttgggggtgcctgccggcgctgacccctggtcctctctctgaacctcagattgaggagatctttaagaaagctggccaccccttcatgtggaaccagcacctgggctacgtgctcacctgcccatccaacctgggcactgggctgcgtggaggcgtgcatgtgaagctggcgcacctgagcaagcaccccaagttcgaggagatcctcacccgcctgcgtctgcagaagaggggtacaggtacggctcccacatcctttgcaccacgacggagggggagcgagggccctttgcagggatgcaaggagaggtgcgacatccctgcgagttctggggatccggcaagggctgaccctctgtgagcttgcatttcttcctctgtaaaatgggtgtcaatatgcctatgtgataggagtcaatgtgggattttcatgattatataatatgcattgcacatgcccttggacctccctcggggcctcagtttccccatttataaagcgaggagtggtcaggattcgaccctaggccgcctggctccagaacccactctcttacccactatactatgtttgataactgagtggcgtggtaccccatctgaagtcagagactcgtgagctcacgaatgaacaggcattacctgtgtgcaggtaacctgttacacagtaggcactcactacatgcggctgttgcatttggcacgggggccacgtcccagcccctctgcaccttcgctgcctcttcacgcccctgtcccttgctcccacaggtggcgtggacacagctgccgtgggctcagtatttgacgtgtccaacgctgatcggctgggctcgtccgaagtagaacaggtgcagctggtggtggatggtgtgaagctcatggtggaaatggagaagaagttggagaaaggccagtccatcgacgacatgatccccgcccagaagtaggcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggccttagctttgtgcgatagttattgagggaggacgtgcaattggccactgtgtggtctcagagtggtttgtcctgtctaggcatgtactaaacatgtcttatcccgggtctaaaatgaggcaaagttaggtcacagaatctagtccaaaaattagaaggctctatggggaatctgagacccagagagacactgagcccagtgtcactcacccaaagcatgggccctttccaactatggctttgaattagtcatgctcagatctcaaacacgaggtccacgctggggaggaatgcagccaaacgaaatgtctgaaacttgaatttagcccaacggtctccagttttgagacctcccatgcagctcgtcacatctacctatattctgcttctcagtggcctccttctgtcccgtctgacttcatccctctgtagctgctgccagtcccttactttctcaagaacctgccatggctccccatttctcctagaaagcttttctgttattttgcaactcaaggccacactgaaatgcataccatttgctggaggctcacctttccagttctttccccattactcctctgattctttcacacctgacaccctccaattcccacctcaatgtctttgaccaagctgtttccctctccaatctgtgagtctgagcatccccatgaagtctttagggataggccttttggagaactcagtcccaggaaggaacaggaaagaggtgtggtagagcaattggtggagggagaagagttaatagaagtcaagcagtagctgaacatgcattgggtgttgggcacgtcaatcacctccacccagtcaaacaggccaatgtctacgttaacaaggacacagtcggtgggccccaaaagacaaacgttttagggaaaaaaaaaagtgaggtgggagtaaatggaagaaccgacaaagccctcaggaacctaaggtgctttttcaaacctcaagagtcacagagattaagggcccttgtagttcaatctgccctctgttttacagaggggaaattgaggcctagggatgtccccaaaagttcccagggagattactacaaggaaggttaggagagattctaaacattaaggtatgcagaacctgtgagatgctgtgatgttctttactgtactgtccccagttgaagatcccagctccacacccagaggctacatagaccacacacgccagtggggaccttctgagccagccaaagccacagtccagacacctgttaccaggtcctgcccactttgcctcacaggactctcttaaatctatctgcgctctccccatctgtccagccccgacctctttcctgatcttctgacctgtcaacttccctcccttccaccctgcacatagcacaaacccgcccatctctaccctacctacaaccttgccatggctcccccagtgcccttggaagaaagcccaggcccctgtccataaccttcaaggccctactgacgcccctagccccatacctcacagcaattctacacttaccaagtcacatgccatttgttgaggacctgtcctctctcttgctccctggcccttgtatgtgccatttgctgtgcctaaaacgcactcctatccatgtctagctggctccttctcctccaggaagccctccctggctgaggcaggcatcacctctggactcccctataccagccctgcccagtcgctgcctgaggacacatctgcctcctccattggactgtgagctctatgagggcagtgttggggctgtcttgattaccactgtgtccccagcacagaggatgctctcaggaattgaccaagcctcttccctgaagttccctgaacttccatggttccccagtgccatggggagaaagtccagcttattcattttggccccaaaggtgctgtgtgaataagctccgcccttctgtactgcacagacttcaaccactataatctgtccagctcccatg

SEQ ID NO: 4 - protein, 381 aa, synthetic construct
  REGION 1..381: note = CKM amino acid sequence
MPFGNTHNKFKLNYKPEEEYPDLSKHNNHMAKVLTLELYKKLRDKETPSGFTVDDVIQTGVDNPGHPFIMTVGCVAGDEESYEVFKELFDPIISDRHGGYKPTDKHKTDLNHENLKGGDDLDPNYVLSSRVRTGRSIKGYTLPPHCSRGERRAVEKLSVEALNSLTGEFKGKYYPLKSMTEKEQQQLIDDHFLFDKPVSPLLLASGMARDWPDARGIWHNDNKSFLVWVNEEDHLRVISMEKGGNMKEVFRRFCVGLQKIEEIFKKAGHPFMWNQHLGYVLTCPSNLGTGLRGGVHVKLAHLSKHPKFEEILTRLRLQKRGTGGVDTAAVGSVFDVSNADRLGSSEVEQVQLVVDGVKLMVEMEKKLEKGQSIDDMIPAQK

SEQ ID NO: 5 - DNA, 8483 bp, synthetic construct
  misc_feature 1..8483: note = FLNA DNA sequence
  source 1..8483: mol_type = other DNA
gcgtggaggcgcgtcgcgcgcagcggacgccgacagaatccccgaggcgcctggcgcgggcgcgggcgcgaaggcgatccgggcgccaccccgcggtcatcggtcaccggtcgctctcaggaacagcagcgcaacctctgctccctgcctcgcctcccgcgcgcctaggtgcctgcgactttaattaaagggccgtcccctcgccgaggctgcagcaccgcccccccggcttctcgcgcctcaaaatgagtagctcccactctcgggcgggccagagcgcagcaggcgcggctccgggcggcggcgtcgacacgcgggacgccgagatgccggccaccgagaaggacctggcggaggacgcgccgtggaagaagatccagcagaacactttcacgcgctggtgcaacgagcacctgaagtgcgtgagcaagcgcatcgccaacctgcagacggacctgagcgacgggctgcggcttatcgcgctgttggaggtgctcagccagaagaagatgcaccgcaagcacaaccagcggcccactttccgccaaatgcagcttgagaacgtgtcggtggcgctcgagttcctggaccgcgagagcatcaaactggtgtccatcgacagcaaggccatcgtggacgggaacctgaagctgatcctgggcctcatctggaccctgatcctgcactactccatctccatgcccatgtgggacgaggaggaggatgaggaggccaagaagcagacccccaagcagaggctcctgggctggatccagaacaagctgccgcagctgcccatcaccaacttcagccgggactggcagagcggccgggccctgggcgccctggtggacagctgtgccccgggcctgtgtcctgactgggactcttgggacgccagcaagcccgttaccaatgcgcgagaggccatgcagcaggcggatgactggctgggcatcccccaggtgatcacccccgaggagattgtggaccccaacgtggacgagcactctgtcatgacctacctgtcccagttccccaaggccaagctgaagccaggggctcccttgcggcccaaactgaacccgaagaaagcccgtgcctacgggccaggcatcgagcccacaggcaacatggtgaagaagcgggcagagttcactgtggagaccagaagtgctggccagggagaggtgctggtgtacgtggaggacccggccggacaccaggaggaggcaaaagtgaccgccaataacgacaagaaccgcaccttctccgtctggtacgtccccgaggtgacggggactcataaggttactgtgctctttgctggccagcacatcgccaagagccccttcgaggtgtacgtggataagtcacagggtgacgccagcaaagtgacagcccaaggtcccggcctggagcccagtggcaacatcgccaacaagaccacctactttgagatctttacggcaggagctggcacgggcgaggtcgaggttgtgatccaggaccccatgggacagaagggcacggtagagcctcagctggaggcccggggcgacagcacataccgctgcagctaccagcccaccatggagggcgtccacaccgtgcacgtcacgtttgccggcgtgcccatccctcgcagcccctacactgtcactgttggccaagcctgtaacccgagtgcctgccgggcggttggccggggcctccagcccaagggtgtgcgggtgaaggagacagctgacttcaaggtgtacacaaagggcgctggcagtggggagctgaaggtcaccgtgaagggccccaagggagaggagcgcgtgaagcagaaggacctgggggatggcgtgtatggcttcgagtattaccccatggtccctggaacctatatcgtcaccatcacgtggggtggtcagaacatcgggcgcagtcccttcgaagtgaaggtgggcaccgagtgtggcaatcagaaggtacgggcctggggccctgggctggagggcggcgtcgttggcaagtcagcagactttgtggtggaggctatcggggacgacgtgggcacgctgggcttctcggtggaagggccatcgcaggctaagatcgaatgtgacgacaagggcgacggctcctgtgatgtgcgctactggccgcaggaggctggcgagtatgccgttcacgtgctgtgcaacagcgaagacatccgcctcagccccttcatggctgacatccgtgacgcgccccaggacttccacccagacagggtgaaggcacgtgggcctggattggagaagacaggtgtggccgtcaacaagccagcagagttcacagtggatgccaagcacggtggcaaggccccacttcgggtccaagtccaggacaatgaaggctgccctgtggaggcgttggtcaaggacaacggcaatggcacttacagctgctcctacgtgcccaggaagccggtgaagcacacagccatggtgtcctggggaggcgtcagcatccccaacagccccttcagggtgaatgtgggagctggcagccaccccaacaaggtcaaagtatacggccccggagtagccaagacagggctcaaggcccacgagcccacctacttcactgtggactgcgccgaggctggccagggggacgtcagcatcggcatcaagtgtgcccctggagtggtaggccccgccgaagctgacatcgacttcgacatcatccgcaatgacaatgacaccttcacggtcaagtacacgccccggggggctggcagctacaccattatggtcctctttgctgaccaggccacgcccaccagccccatccgagtcaaggtggagccctctcatgacgccagtaaggtgaaggccgagggccctggcctcagtcgcactggtgtcgagcttggcaagcccacccacttcacagtaaatgccaaagctgctggcaaaggcaagctggacgtccagttctcaggactcaccaagggggatgcagtgcgagatgtggacatcatcgaccaccatgacaacacctacacagtcaagtacacgcctgtccagcagggtccagtaggcgtcaatgtcacttatggaggggatcccatccctaagagccctttctcagtggcagtatctccaagcctggacctcagcaagatcaaggtgtctggcctgggagagaaggtggacgttggcaaagaccaggagttcacagtcaaatcaaagggtgctggtggtcaaggcaaagtggcatccaagattgtgggcccctcgggtgcagcggtgccctgcaaggtggagccaggcctgggggctgacaacagtgtggtgcgcttcctgccccgtgaggaagggccctatgaggtggaggtgacctatgacggcgtgcccgtgcctggcagcccctttcctctggaagctgtggcccccaccaagcctagcaaggtgaaggcgtttgggccggggctgcagggaggcagtgcgggctcccccgcccgcttcaccatcgacaccaagggcgccggcacaggtggcctgggcctgacggtggagggcccctgtgaggcgcagctcgagtgcttggacaatggggatggcacatgttccgtgtcctacgtgcccaccgagcccggggactacaacatcaacatcctcttcgctgacacccacatccctggctccccattcaaggcccacgtggttccctgctttgacgcatccaaagtcaagtgctcaggccccgggctggagcgggccaccgctggggaggtgggccaattccaagtggactgctcgagcgcgggcagcgcggagctgaccattgagatctgctcggaggcggggcttccggccgaggtgtacatccaggaccacggtgatggcacgcacaccattacctacattcccctctgccccggggcctacaccgtcaccatcaagtacggcggccagcccgtgcccaacttccccagcaagctgcaggtggaacctgcggtggacacttccggtgtccagtgctatgggcctggtattgagggccagggtgtcttccgtgaggccaccactgagttcagtgtggacgcccgggctctgacacagaccggagggccgcacgtcaaggcccgtgtggccaacccctcaggcaacctgacggagacctacgttcaggaccgtggcgatggcatgtacaaagtggagtacacgccttacgaggagggactgcactccgtggacgtgacctatgacggcagtcccgtgcccagcagccccttccaggtgcccgtgaccgagggctgcgacccctcccgggtgcgtgtccacgggccaggcatccaaagtggcaccaccaacaagcccaacaagttcactgtggagaccaggggagctggcacgggcggcctgggcctggctgtagagggcccctccgaggccaagatgtcctgcatggataacaaggacggcagctgctcggtcgagtacatcccttatgaggctggcacctacagcctcaacgtcacctatggtggccatcaagtgccaggcagtcctttcaaggtccctgtgcatgatgtgacagatgcgtccaaggtcaagtgctctgggcccggcctgagcccaggcatggttcgtgccaacctccctcagtccttccaggtggacacaagcaaggctggtgtggccccattgcaggtcaaagtgcaagggcccaaaggcctggtggagccagtggacgtggtagacaacgctgatggcacccagaccgtcaattatgtgcccagccgagaagggccctacagcatctcagtactgtatggagatgaagaggtaccccggagccccttcaaggtcaaggtgctgcctactcatgatgccagcaaggtgaaggccagtggccccgggctcaacaccactggcgtgcctgccagcctgcccgtggagttcaccatcgatgcaaaggacgccggggagggcctgctggctgtccagatcacggatcccgaaggcaagccgaagaagacacacatccaagacaaccatgacggcacgtatacagtggcctacgtgccagacgtgacaggtcgctacaccatcctcatcaagtacggtggtgacgagatccccttctccccgtaccgcgtgcgtgccgtgcccaccggggacgccagcaagtgcactgtcacaggtgctggcatcggccccaccattcagattggggaggagacggtgatcactgtggacactaaggcggcaggcaaaggcaaagtgacgtgcaccgtgtgcacgcctgatggctcagaggtggatgtggacgtggtggagaatgaggacggcactttcgacatcttctacacggccccccagccgggcaaatacgtcatctgtgtgcgctttggtggcgagcacgtgcccaacagccccttccaagtgacggctctggctggggaccagccctcggtgcagccccctctacggtctcagcagctggccccacagtacacctacgcccagggcggccagcagacttgggccccggagaggcccctggtgggtgtcaatgggctggatgtgaccagcctgaggccctttgaccttgtcatccccttcaccatcaagaagggcgagatcacaggggaggttcggatgccctcaggcaaggtggcgcagcccaccatcactgacaacaaagacggcaccgtgaccgtgcggtatgcacccagcgaggctggcctgcacgagatggacatccgctatgacaacatgcacatcccaggaagccccttgcagttctatgtggattacgtcaactgtggccatgtcactgcctatgggcctggcctcacccatggagtagtgaacaagcctgccaccttcaccgtcaacaccaaggatgcaggagaggggggcctgtctctggccattgagggcccgtccaaagcagaaatcagctgcactgacaaccaggatgggacatgcagcgtgtcctacctgcctgtgctgccgggggactacagcattctagtcaagtacaatgaacagcacgtcccaggcagccccttcactgctcgggtcacaggtgacgactccatgcgtatgtcccacctaaaggtcggctctgctgccgacatccccatcaacatctcagagacggatctcagcctgctgacggccactgtggtcccgccctcgggccgggaggagccctgtttgctgaagcggctgcgtaatggccacgtggggatttcattcgtgcccaaggagacgggggagcacctggtgcatgtgaagaaaaatggccagcacgtggccagcagccccatcccggtggtgatcagccagtcggaaattggggatgccagtcgtgttcgggtctctggtcagggccttcacgaaggccacacctttgagcctgcagagtttatcattgatacccgcgatgcaggctatggtgggctcagcctgtccattgagggccccagcaaggtggacatcaacacagaggacctggaggacgggacgtgcagggtcacctactgccccacagagccaggcaactacatcatcaacatcaagtttgccgaccagcacgtgcctggcagccccttctctgtgaaggtgacaggcgagggccgggtgaaagagagcatcacccgcaggcgtcgggctccttcagtggccaacgttggtagtcattgtgacctcagcctgaaaatccctgaaattagcatccaggatatgacagcccaggtgaccagcccatcgggcaagacccatgaggccgagatcgtggaaggggagaaccacacctactgcatccgctttgttcccgctgagatgggcacacacacagtcagcgtgaagtacaagggccagcacgtgcctgggagccccttccagttcaccgtggggcccctaggggaagggggagcccacaaggtccgagctgggggccctggcctggagagagctgaagctggagtgccagccgaattcagtatctggacccgggaagctggtgctggaggcctggccattgctgtcgagggccccagcaaggctgagatctcttttgaggaccgcaaggacggctcctgtggtgtggcttatgtggtccaggagccaggtgactacgaagtctcagtcaagttcaacgaggaacacattcccgacagccccttcgtggtgcctgtggcttctccgtctggcgacgcccgccgcctcactgtttctagccttcaggagtcagggctaaaggtcaaccagccagcctcttttgcagtcagcctgaacggggccaagggggcgatcgatgccaaggtgcacagcccctcaggagccctggaggagtgctatgtcacagaaattgaccaagataagtatgctgtgcgcttcatccctcgggagaatggcgtttacctgattgacgtcaagttcaacggcacccacatccctggaagccccttcaagatccgagttggggagcctgggcatggaggggacccaggcttggtgtctgcttacggagcaggtctggaaggcggtgtcacagggaacccagctgagttcgtcgtgaacacgagcaatgcgggagctggtgccctgtcggtgaccattgacggcccctccaaggtgaagatggattgccaggagtgccctgagggctaccgcgtcacctatacccccatggcacctggcagctacctcatctccatcaagtacggcggcccctaccacattgggggcagccccttcaaggccaaagtcacaggcccccgtctcgtcagcaaccacagcctccacgagacatcatcagtgtttgtagactctctgaccaaggccacctgtgccccccagcatggggccccgggtcctgggcctgctgacgccagcaaggtggtggccaagggcctggggctgagcaaggcctacgtaggccagaagagcagcttcacagtagactgcagcaaagcaggcaacaacatgctgctggtgggggttcatggcccaaggaccccctgcgaggagatcctggtgaagcacgtgggcagccggctctacagcgtgtcctacctgctcaaggacaagggggagtacacactggtggtcaaatggggggacgagcacatcccaggcagcccctaccgcgttgtggtgccctgagtctggggcccgtgccagccggcagcccccaagcctgccccgctacccaagcagccccgccctcttcccctcaaccccggcccaggccgccctggccgcccgcctgtcactgcagccgcccctgccctgtgccgtgctgcgctcacctgcctccccagccagccgctgacctctcggctttcacttgggcagagggagccatttggtggcgctgcttgtcttctttggttctgggaggggtgagggatgggggtcctgtacacaaccacccactagttctcttctccagccaagaggaataaagttttgcttccattc

SEQ ID NO: 6 - protein, 2647 aa, synthetic construct
  REGION 1..2647: note = FLNA amino acid sequence
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 7 - DNA, 3479 bp, synthetic construct
  misc_feature 1..3479: note = ITGA2B DNA sequence
  source 1..3479: mol_type = other DNA
acttcctggcaattctagccaccatgagtccaggggctatagccctttgctctgcccgttgctcagcaagttacttggggttccagtttgataagaaaagacttcctgtggaggaatctgaagggaaggaggaggagctggcccattcctgcctgggaggttgtggaagaaggaagatggccagagctttgtgtccactgcaagccctctggcttctggagtgggtgctgctgctcttgggaccttgtgctgcccctccagcctgggccttgaacctggacccagtgcagctcaccttctatgcaggccccaatggcagccagtttggattttcactggacttccacaaggacagccatgggagagtggccatcgtggtgggcgccccgcggaccctgggccccagccaggaggagacgggcggcgtgttcctgtgcccctggagggccgagggcggccagtgcccctcgctgctctttgacctccgtgatgagacccgaaatgtaggctcccaaactttacaaaccttcaaggcccgccaaggactgggggcgtcggtcgtcagctggagcgacgtcattgtggcctgcgccccctggcagcactggaacgtcctagaaaagactgaggaggctgagaagacgcccgtaggtagctgctttttggctcagccagagagcggccgccgcgccgagtactccccctgtcgcgggaacaccctgagccgcatttacgtggaaaatgattttagctgggacaagcgttactgtgaagcgggcttcagctccgtggtcactcaggccggagagctggtgcttggggctcctggcggctattatttcttaggtctcctggcccaggctccagttgcggatattttctcgagttaccgcccaggcatccttttgtggcacgtgtcctcccagagcctctcctttgactccagcaacccagagtacttcgacggctactgggggtactcggtggccgtgggcgagttcgacggggatctcaacactacagaatatgtcgtcggtgcccccacttggagctggaccctgggagcggtggaaattttggattcctactaccagaggctgcatcggctgcgcggagagcagatggcgtcgtattttgggcattcagtggctgtcactgacgtcaacggggatgggaggcatgatctgctggtgggcgctccactgtatatggagagccgggcagaccgaaaactggccgaagtggggcgtgtgtatttgttcctgcagccgcgaggcccccacgcgctgggtgcccccagcctcctgctgactggcacacagctctatgggcgattcggctctgccatcgcacccctgggcgacctcgaccgggatggctacaatgacattgcagtggctgccccctacgggggtcccagtggccggggccaagtgctggtgttcctgggtcagagtgaggggctgaggtcacgtccctcccaggtcctggacagccccttccccacaggctctgcctttggcttctcccttcgaggtgccgtagacatcgatgacaacggatacccagacctgatcgtgggagcttacggggccaaccaggtggctgtgtacagagctcagccagtggtgaaggcctctgtccagctactggtgcaagattcactgaatcctgctgtgaagagctgtgtcctacctcagaccaagacacccgtgagctgcttcaacatccagatgtgtgttggagccactgggcacaacattcctcagaagctatccctaaatgccgagctgcagctggaccggcagaagccccgccagggccggcgggtgctgctgctgggctctcaacaggcaggcaccaccctgaacctggatctgggcggaaagcacagccccatctgccacaccaccatggccttccttcgagatgaggcagacttccgggacaagctgagccccattgtgctcagcctcaatgtgtccctaccgcccacggaggctggaatggcccctgctgtcgtgctgcatggagacacccatgtgcaggagcagacacgaatcgtcctggactgtggggaagatgacgtatgtgtgccccagcttcagctcactgccagcgtgacgggctccccgctcctagttggggcagataatgtcctggagctgcagatggacgcagccaacgagggcgagggggcctatgaagcagagctggccgtgcacctgccccagggcgcccactacatgcgggccctaagcaatgtcgagggctttgagagactcatctgtaatcagaagaaggagaatgagaccagggtggtgctgtgtgagctgggcaaccccatgaagaagaacgcccagataggaatcgcgatgttggtgagcgtggggaatctggaagaggctggggagtctgtgtccttccagctgcagatacggagcaagaacagccagaatccaaacagcaagattgtgctgctggacgtgccggtccgggcagaggcccaagtggagctgcgagggaactcctttccagcctccctggtggtggcagcagaagaaggtgagagggagcagaacagcttggacagctggggacccaaagtggagcacacctatgagctccacaacaatggccctgggactgtgaatggtcttcacctcagcatccaccttccgggacagtcccagccctccgacctgctctacatcctggatatacagccccaggggggccttcagtgcttcccacagcctcctgtcaaccctctcaaggtggactgggggctgcccatccccagcccctcccccattcacccggcccatcacaagcgggatcgcagacagatcttcctgccagagcccgagcagccctcgaggcttcaggatccagttctcgtaagctgcgactcggcgccctgtactgtggtgcagtgtgacctgcaggagatggcgcgcgggcagcgggccatggtcacggtgctggccttcctgtggctgcccagcctctaccagaggcctctggatcagtttgtgctgcagtcgcacgcatggttcaacgtgtcctccctcccctatgcggtgcccccgctcagcctgccccgaggggaagctcaggtgtggacacagctgctccgggccttggaggagagggccattccaatctggtgggtgctggtgggtgtgctgggtggcctgctgctgctcaccatcctggtcctggccatgtggaaggtcggcttcttcaagcggaaccggccacccctggaagaagatgatgaagagggggagtgatggtgcagcctacactattctagcaggagggttgggcgtgctacctgcaccgccccttctccaacaagttgcctccaagctttgggttggagctgttccattgggtcctcttggtgtcgtttccctcccaacagagctgggctaccccccctcctgctgcctaataaagagactgagccctga

SEQ ID NO: 8 - protein, 1039 aa, synthetic construct
  REGION 1..1039: note = ITGA2B amino acid sequence
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 9 - DNA, 759 bp, synthetic construct
  misc_feature 1..759: note = ORM2 DNA sequence
  source 1..759: mol_type = other DNA
agcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccggatcactggcaagtggttttatatcgcatcggcctttcgaaacgaggagtacaataagtcggttcaggagatccaagcaaccttcttttactttacccccaacaagacagaggacacgatctttctcagagagtaccagacccgccagaaccagtgcttctataactccagttacctgaatgtccagcgggagaatgggaccgtctccagatacgagggaggccgagaacatgttgctcacctgctgttccttagggacaccaagaccttgatgtttggttcctacctggacgatgagaagaactgggggctgtctttctatgctgacaagccagagacgaccaaggagcaactgggagagttctacgaagctctcgactgcttgtgcattcccaggtcagatgtcatgtacaccgactggaaaaaggataagtgtgagccactggagaagcagcacgagaaggagaggaaacaggaggagggggaatcctagcaggacacagccttggatcaggacagagacttgggggccatcctgcccctccaacccgacatgtgtacctcagctttttccctcacttgcatcaataaagcttctgtgtttggaaca

SEQ ID NO: 10 - protein, 201 aa, synthetic construct
  REGION 1..201: note = ORM2 amino acid sequence
MALSWVLTVLSLLPLLEAQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLCIPRSDVMYTDWKKDKCEPLEKQHEKERKQEEGES

SEQ ID NO: 11 - DNA, 2100 bp, synthetic construct
  misc_feature 1..2100: note = PLTP DNA sequence
  source 1..2100: mol_type = other DNA
tttccttccctctccctgggcttgggtctcccacttgtccagacagcggccgggcttgtcacggggctctgtgcagccttttccactctcccggctgccagcgtcccgccccgtcccctcccagcccccaagggaggaggggagagctgcagagaggaggaggggtcggggaggccggctttataaaggcggctggaacaaccctgcccgccagaccccgtcgcccggatcccctgagctgcccgccatcccacgtgaccgcgccgccccccagctccaccgctgagcccgctcgccatggccctcttcggggccctcttcctagcgctgctggcaggcgcacatgcagagttcccaggctgcaagatccgcgtcacctccaaggcgctggagctggtgaagcaggaggggctgcgctttctggagcaagagctggagactatcaccattccggacctgcggggcaaagaaggccacttctactacaacatctctgaggtgaaggtcacagagctgcaactgacatcttccgagctcgatttccagccacagcaggagctgatgcttcaaatcaccaatgcctccttggggctgcgcttccggagacagctgctctactggttcttctatgatgggggctacatcaacgcctcagctgagggtgtgtccatccgcactggtctggagctctcccgggatcccgctggacggatgaaagtgtccaatgtctcctgccaggcctctgtctccagaatgcacgcggccttcgggggaaccttcaagaaggtgtatgattttctctccacgttcatcacctcagggatgcgcttcctcctcaaccagcagatctgccctgtcctctaccacgcagggacggtcctgctcaactccctcctggacaccgtgcctgtgcgcagttctgtggacgagcttgttggcattgactattccctcatgaaggatcctgtggcttccaccagcaacctggacatggacttccggggggccttcttccccctgactgagaggaactggagcctccccaaccgggcagtggagccccagctgcaggaggaagagcggatggtgtatgtggccttctctgagttcttcttcgactctgccatggagagctacttccgggcgggggccctgcagctgttgctggtgggggacaaggtgccccacgacctggacatgctgctgagggccacctactttgggagcattgtcctgctgagcccagcagtgattgactccccattgaagctggagctgcgggtcctggccccaccgcgctgcaccatcaagccctctggcaccaccatctctgtcactgctagcgtcaccattgccctggtcccaccagaccagcctgaggtccagctgtccagcatgactatggacgcccgtctcagcgccaagatggctctccgggggaaggccctgcgcacgcagctggacctgcgcaggttccgaatctattccaaccattctgcactggagtcgctggctctgatcccattacaggcccctctgaagaccatgctgcagattggggtgatgcccatgctcaatgagcggacctggcgtggggtgcagatcccactacctgagggcatcaactttgtgcatgaggtggtgacgaaccatgcgggattcctcaccatcggggctgatctccactttgccaaagggctgcgagaggtgattgagaagaaccggcctgctgatgtcagggcgtccactgcccccacaccgtccacagcagctgtctgagccctcaatccccaagctggcagctgtcattcaggaccccaacccctctcagcccctcttttcccacattcatagcctgtagtgccccctctaacccccagtgccacagagaagacgggatttgaagctgtacccaatttaattccataatcaatctatcaattacagtccgtccaccacctccctgtgggctgtcctgagctctgttgggttcctgggatggaatcagtgcatcataaagggcattctttaagcagagaaggggccaggccaccccattcaggaactgctgcgggaataaagtgctaacttgcccccagg

SEQ ID NO: 12 - protein, 493 aa, synthetic construct
  REGION 1..493: note = PLTP amino acid sequence
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 13 - DNA, 1357 bp, synthetic construct
  misc_feature 1..1357: note = HP DNA sequence
  source 1..1357: mol_type = other DNA
actggaaaagatagtgaccttaccagggccaaagtttgtagacacaggaattacgaaatggagaagggggagaagtgagctagtggcagcataaaaagaccagcagatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgaccca

SEQ ID NO: 14 - protein, 406 aa, synthetic construct
  REGION 1..406: note = HP amino acid sequence
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 15 - DNA, 2559 bp, synthetic construct
  misc_feature 1..2559: note = QSOX1 DNA sequence
  source 1..2559: mol_type = other DNA
gcgcagcgccgaggatgaggaggtgcaacagcggctccgggccgccgccgtcgctgctgctgctgctgctgtggctgctcgcggttcccggcgctaacgcggccccgcggtcggcgctctattcgccttccgacccgctgacgctgctgcaggcggacacggtgcgcggcgcggtgctgggctcccgcagcgcctgggccgtggagttcttcgcctcctggtgcggccactgcatcgccttcgccccgacgtggaaggcgctggccgaagacgtcaaagcctggaggccggccctgtatctcgccgccctggactgtgctgaggagaccaacagtgcagtctgcagagacttcaacatccctggcttcccgactgtgaggttcttcaaggcctttaccaagaacggctcaggagcagtatttccagtggctggtgctgacgtgcagacgctgcgggagaggctcattgacgccctggagtcccatcatgacacgtggcccccagcctgtcccccactggagcctgccaagctggaggagattgatggattctttgcgagaaataacgaagagtacctggctctgatctttgaaaagggaggctcctacctggctagagaggtggctctggacctgtcccagcacaaaggcgtggcggtgcgcagggtgctgaacacagaggccaatgtggtgagaaagtttggtgtcaccgacttcccctcttgctacctgctgttccggaatggctctgtctcccgagtccccgtgctcatggaatccaggtccttctataccgcttacctgcagagactctctgggctcaccagggaggctgcccagaccacagttgcaccaaccactgctaacaagatagctcccactgtttggaaattggcagatcgctccaagatctacatggctgacctggaatctgcactgcactacatcctgcggatagaagtgggcaggttcccggtcctggaagggcagcgcctggtggccctgaaaaagtttgtggcagtgctggccaagtatttccctggccggcccttagtccagaacttcctgcactccgtgaatgaatggctcaagaggcagaagagaaataaaattccctacagtttctttaaaactgccctggacgacaggaaagagggtgccgttcttgccaagaaggtgaactggattggctgccaggggagtgagccgcatttccggggctttccctgctccctgtgggttcttttccacttcttgactgtgcaggcagctcggcaaaatgtagaccactcacaggaagcagccaaggccaaggaggtcctcccagccatccgaggctacgtgcactacttcttcggctgccgagactgcgctagccacttcgagcagatggctgctgcctccatgcaccgggtggggagtcccaacgccgctgtcctctggctctggtctagccacaacagggtcaatgctcgccttgcaggtgcccccagcgaggacccccagttccccaaggtgcagtggccaccccgtgaactttgttctgcctgccacaatgaacgcctggatgtgcccgtgtgggacgtggaagccaccctcaacttcctcaaggcccacttctccccaagcaacatcatcctggacttccctgcagctgggtcagctgcccggagggatgtgcagaatgtggcagccgccccagagctggcgatgggagccctggagctggaaagccggaattcaactctggaccctgggaagcctgagatgatgaagtcccccacaaacaccaccccacatgtgccggctgagggacctgagcttatttgaagtcctgcctcattctcactggagcctcagtctctcctgcttggtcttggccctcaactggggcaagtgaagccagaggagggtcccccagctgggtgggctggaatggaactcctcactagctgctggggctccgcccaccctgctcccttccggacaatgaagaagcctttgcaccctgggaggaaggaccaccccgggccctctatgcctggccagcctccagctcctcagacctcctgggtggggtttggcttcagggtggggtttggaagcttctggaagtcgtgctggtctcccaggtgaggcaagccatggttgctgggctgtagggtgagtggcttgcttggtgggacctgacgagttggtggcatgggaaggatgtgggtctctagtgccttgccctggcttagctgcaggagaagatggctgctttcacttccccccattgagcgctgctccctctgagcctggtcttttgtccttttttattttggtctccaagatgaatgctcatctttggagggtgccaggtagaagctagggaggggagtgtcttctctctccaggtttcaccttccagtgtgcagaagttagaagggtctggcgggggcagtgccttacacatgcttgattcccacgctaccccctgccttgggaggtgtgtggaataaattatttttgttaaggcaaaaaaaaaaaaaaaaaaaaaaaaaaaaaaaaaaaaaaa

SEQ ID NO: 16 - protein, 329 aa, synthetic construct
  REGION 1..329: note = QSOX1 amino acid sequence
MRRCNSGSGPPPSLLLLLLWLLAVPGANAAPRSALYSPSDPLTLLQADTVRGAVLGSRSAWAVEFFASWCGHCIAFAPTWKALAEDVKAWRPALYLAALDCAEETNSAVCRDFNIPGFPTVRFFKAFTKNGSGAVFPVAGADVQTLRERLIDALESHHDTWPPACPPLEPAKLEEIDGFFARNNEEYLALIFEKGGSYLAREVALDLSQHKGVAVRRVLNTEANVVRKFGVTDFPSCYLLFRNGSVSRVPVLMESRSFYTAYLQRLSGLTREAAQTTVAPTTANKIAPTVWKLADRSKIYMADLESALHYILRIEVGRFPVLEGQRLVA

SEQ ID NO: 17 - DNA, 5042 bp, synthetic construct
  misc_feature 1..5042: note = TGM2 DNA sequence
  source 1..5042: mol_type = other DNA
acagtggatccggaagcacaggagaccaagagaccagagataccaggatgagagataggacccctggttgccagtgccagccgccagtggtcgcacttggagggtctcgccgccagtggaaggagccaccgcccccgcccgaccatggccgaggagctggtcttagagaggtgtgatctggagctggagaccaatggccgagaccaccacacggccgacctgtgccgggagaagctggtggtgcgacggggccagcccttctggctgaccctgcactttgagggccgcaactacgaggccagtgtagacagtctcaccttcagtgtcgtgaccggcccagcccctagccaggaggccgggaccaaggcccgttttccactaagagatgctgtggaggagggtgactggacagccaccgtggtggaccagcaagactgcaccctctcgctgcagctcaccaccccggccaacgcccccatcggcctgtatcgcctcagcctggaggcctccactggctaccagggatccagctttgtgctgggccacttcattttgctcttcaacgcctggtgcccagcggatgctgtgtacctggactcggaagaggagcggcaggagtatgtcctcacccagcagggctttatctaccagggctcggccaagttcatcaagaacataccttggaattttgggcagtttgaagatgggatcctagacatctgcctgatccttctagatgtcaaccccaagttcctgaagaacgccggccgtgactgctcccgccgcagcagccccgtctacgtgggccgggtggtgagtggcatggtcaactgcaacgatgaccagggtgtgctgctgggacgctgggacaacaactacggggacggcgtcagccccatgtcctggatcggcagcgtggacatcctgcggcgctggaagaaccacggctgccagcgcgtcaagtatggccagtgctgggtcttcgccgccgtggcctgcacagtgctgaggtgcctgggcatccctacccgcgtcgtgaccaactacaactcggcccatgaccagaacagcaaccttctcatcgagtacttccgcaatgagtttggggagatccagggtgacaagagcgagatgatctggaacttccactgctgggtggagtcgtggatgaccaggccggacctgcagccggggtacgagggctggcaggccctggacccaacgccccaggagaagagcgaagggacgtactgctgtggcccagttccagttcgtgccatcaaggagggcgacctgagcaccaagtacgatgcgccctttgtctttgcggaggtcaatgccgacgtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtctttgcccacatcaccaacaacaccgctgaggagtacgtctgccgcctcctgctctgtgcccgcaccgtcagctacaatgggatcttggggcccgagtgtggcaccaagtacctgctcaacctcaacctggagcctttctctgagaagagcgttcctctttgcatcctctatgagaaataccgtgactgccttacggagtccaacctcatcaaggtgcgggccctcctcgtggagccagttatcaacagctacctgctggctgagagggacctctacctggagaatccagaaatcaagatccggatccttggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctgctgccgctccacatgggcctccacaagctggtggtgaacttcgagagcgacaagctgaaggctgtgaagggcttccggaatgtcatcattggccccgcctaagggacccctgctcccagcctgctgagagcccccaccttgatcccaatccttatcccaagctagtgagcaaaatatgccccttcttgggccccagaccccagggcagggtgggcagcctatgggggctctcggaaatggaatgtgcccctggcccatctcagcctcctgagcctgtgggtccccactcaccccctttgctgtgaggaatgctctgtgccagaaacagtgggagccctgaccttggctgactggggctggggtgagagaggaaagacctacattccctctcctgcccagatgccctttggaaagccattgaccacccaccatattgtttgatctacttcatagctccttggagcaggcaaaaaagggacagcatgccccttggctggatcagggaatccagctccctagactgcatcccgtacctcttcccatgactgcacccagctccaggggcccttgggacagccagagctgggtggggacagtgataggcccaaggtcccctccacatcccagcagcccaagcttaatagccctccccctcaacctcaccattgtgaagcacctactatgtgctgggtgcctcccacacttgctggggctcacggggcctccaacccatttaatcaccatgggaaactgttgtgggcgctgcttccaggataaggagactgaggcttagagagaggaggcagccccctccacaccagtggcctcgtggttattagcaaggctgggtaatgtgaaggcccaagagcagagtctgggcctctgactctgagtccactgctccatttataaccccagcctgacctgagactgtcggagaggctgtctggggcctttatcaaaaaaagactcagccaagacaaggaggtagagaggggactgggggactgggagtcagagccctggctgggttcaggtcccacgtctggccaggcactgccttctcctctctgggcctttgtttccttgttggtcagaggagtgattgaaccagctcatctccaaggatcctctccactccatgtttgcaatgcttttatatggcccagccttgtaaataaccacaaggtccactccctgctccacgaagccttaagccataggcccaggatatttctgagagtgaaaccatgactgtgaccaccttctgtccccagccctgtcctggttccttcctatgcccaggtaccacccttcagaccccagttctaggggagaagagccctggacacccctgctctacccatgagcctgcccgctgcaatgcctagacttcccaacagccttagctgccagtgctggtcactaaccaacaaggttggcaccccagctaccccttctttgcagggctaaggcccccaaacatagcccctgccccggaggaagcttggggaacccatgagttgtcagctttgactttatctcctgctctttctacatgactgggcctcccttgggctggaagaattggggattctctattggaggtgagatcacagcctccagggccccccaaatcccagggaaggacttggagagaatcatgctgttgcatttagaactttctgctttgcacaggaaagagtcacacaattaatcaacatgtatattttctctatacatagagctctatttctctacggttttataaaagccttgggttccaaccaggcagtagatgtgcttctgaaccgcaaggagcaaacactgaaataaaatagtttatttttcacactcaaactaggtctggtctgttcctttatgagtttggagggagatgggtaggtgagctcgctcacacctggacccccacttcctgcctaggagtctttcctgagcctcagccctgtccctgcgtttaaactttactggcattcaaggtcctacacaggctggctccatctccggccccttccaccaacagtctgtttttgggccacaattaacttcctgtatccccagaacaccctgcgtagttgtctacctcttggcttttgtgtatgtagttccctgagcctggagtgcccctccttttctttccctgcttggcaaactcctactcatcctataatgcttaacaccaaagtcacctcttctgtgaaaccttccttgatatcttttcgggtccccagagtcctggaatatggaccaattcaaacatttatcacagtaaattttacttccacgttggcctatctgtccccttctccatcctatctgaagccctcgaggcagggaccacgtatctcttaaatacactaataaatgctgaattggtaaatgaatgtttcctaactcaactcccacaagacctgtgctgtcccatagaaataatacatgagccacaaatgcaagccacatatgtaatttagaattttctagtgaccacattgaaaacagtaaaaaagccataggtggaattagctttaataatgtattttatttaatccaatatatccaaaacattatttcaacatgtaatcaatataagaattagtaaggtatttgaccttcttatttttgtactcaatctttgaaatgtggtgtgtgttttatactgacagcaggtctcaatttgaactagctgcatttcaagtgctcggtagccagtctggctagtactggatgaagcaggtctagactgcgattccgtggatgtgcagacttggtcattcatctcattccccaccatcgaagcactcatgagtatctgttgaatgaatagatgatggggacagtccccacccttgggaagtcatgtagaaagacatgacatgcacataaaaaataaatcaaaataaaaacatgacatcacg

SEQ ID NO: 18 - protein, 687 aa, synthetic construct
  REGION 1..687: note = TGM2 amino acid sequence
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 19 - DNA, 9060 bp, synthetic construct
  misc_feature 1..9060: note = FLNC DNA sequence
  source 1..9060: mol_type = other DNA
gcccttcccgagcaccgctccggccctggagggagagagagccagagagcggccgagcgcctaggaggcccgccgagcctcgccgagccccgccagccccggcgcgagagaagttggagaggagagcagcgcagcgcagcgagtcccgtggtcgcgccccaacagcgcccgacagcccccgatagcccaaaccgcggccctagccccggccgcacccccagcccgcgccagcatgatgaacaacagcggctactcagacgccggcctcggcctgggcgatgagacagacgagatgccgtccacggagaaggacctggcggaggacgcgccgtggaagaagatccagcagaacacattcacgcgctggtgcaatgagcacctcaagtgcgtgggcaagcgcctgaccgacctgcagcgcgacctcagcgacgggctccggctcatcgcgctgctcgaggtgctcagccagaagcgcatgtaccgcaagttccatccgcgccccaacttccgccaaatgaagctggagaacgtgtccgtggccctcgagttcctcgagcgcgagcacatcaagctcgtgtccatagacagcaaggccatcgtggatgggaacctgaagctgatcctgggcctgatctggacgctgatcctgcactactccatctccatgcccatgtgggaggatgaagatgatgaggatgcccgcaaacagacgcccaagcagcggctgcttggctggatccagaacaaggtgccccagctgcccatcaccaacttcaaccgtgactggcaggacggcaaagctctgggcgccctggtggacaactgcgcccccggtctctgccccgactgggaggcctgggaccccaaccagcccgtggagaacgcccgggaggccatgcagcaggccgacgactggcttggggtgccccaggtcattgcccctgaggagattgtggaccccaacgtggatgagcattctgttatgacctacctgtcccagttccccaaggccaagctcaaacctggtgcccctgttcgatccaagcagctgaaccccaagaaagccatcgcctatgggcctggcatcgagccacagggcaacaccgtgctgcagcctgcccacttcaccgtgcagacggtggacgcgggcgtgggcgaggtgctggtctacatcgaggaccctgaaggccacaccgaggaggctaaggtggttcccaacaatgacaaggatcgcacctatgctgtctcctatgtgcccaaggtcgctgggttacacaaggtgaccgtgctctttgctggccagaacattgaacgcagtccctttgaggtgaacgtgggcatggccctgggagatgccaacaaggtgtcagcccgtggccctggcctggaacctgtgggcaatgtggccaacaaacccacctactttgacatctacactgcgggggccggcactggcgatgttgctgtggtgatcgtggacccacagggccggcgggacacagtggaggtggccctggaggacaagggtgacagcacgttccgctgcacatacagacctgccatggaggggccacataccgtgcatgtggcctttgcgggtgcccccatcacccgcagtcccttccctgtccatgtgtcggaagcctgtaaccccaacgcctgccgcgcctctgggcgaggcctgcagcccaagggtgttcgcgtgaaagaggtggctgacttcaaggtgtttaccaagggtgccggcagcggggagctcaaggtcacggtcaaggggccaaagggcacagaggagccagtgaaggtgcgggaggctggggatggtgtgttcgagtgcgagtactacccggtggtgcctgggaagtatgtggtgaccatcacgtggggcggctacgccatccctcgcagcccctttgaggtacaggtgagcccagaggcaggagtgcaaaaggtccgggcctggggtcctggtttggagactggccaggtgggcaagtcagccgattttgtggtggaagccattggcaccgaggtggggacactgggcttctccatcgaggggccctcacaagccaagatcgaatgtgacgacaagggggatggctcctgcgatgtgcggtactggcccacggagcctggggagtacgctgtgcacgtcatctgtgacgatgaggacatccgagactcacccttcattgcccacatcctgcccgccccacctgactgcttcccagataaggtgaaggcctttgggcctggcctggagcctaccggctgcatcgtggacaagcccgctgagttcaccattgatgctcgtgcagctggcaagggagacctgaagctctatgcccaggacgccgacggctgtcccatcgacatcaaggtgatccccaacggcgacggcaccttccgctgctcctacgtgcccaccaagcccattaagcacaccatcatcatctcctggggaggcgtaaacgtgcccaagagccccttccgggtgaacgtgggcgagggcagccaccccgagcgggtaaaggtgtacggccccggagtggagaagacaggcctcaaggccaatgagcccacctacttcacggtggactgcagcgaggcggggcaaggcgacgtgagcatcggcatcaagtgcgccccaggcgtggtgggccctgcagaggctgacattgacttcgacatcatcaagaatgacaacgacaccttcaccgtcaagtacacgccaccaggggcgggccgctacaccatcatggtgctgtttgccaaccaggagatccccgccagccccttccacatcaaggtggacccatcccacgatgccagcaaagtcaaggccgagggccctgggctgaatcgcacaggtgtggaagtcgggaagcccacccacttcacggtgctgaccaagggagccggcaaggccaagctggatgtgcagtttgcagggacagccaagggcgaggttgtgcgggactttgagatcatagacaaccatgactactcctacactgtcaagtacaccgctgtccagcagggcaacatggcagtgacagtgacttatggcggggaccctgtccccaagagcccctttgtggtgaatgtggcacccccgctggacctcagcaaaatcaaagttcagggccttaatagcaaggtggctgtgggacaggaacaagcattctctgtgaacacacgaggggctggcggtcagggccaactggatgtgcggatgacttcgccctctcgccggcccatcccctgcaagctggagccaggcggtggagcggaagcccaggctgtgcgctacatgcccccggaggaggggccctacaaggtggatatcacctacgatggtcacccggtgcctggcagcccgtttgctgtggagggtgtcctgccccctgatccctccaaggtctgtgcttatggcccgggtctcaagggtggactggtaggcacccccgcgccattctccatcgacaccaagggggctggcacaggtggcctggggctgaccgtagagggcccctgcgaggccaagatcgagtgccaggacaatggtgatggctcatgtgctgtcagctacctgcccacggagcctggcgagtacaccatcaacatcctgtttgctgaggcccacatccctggctcgcccttcaaagccaccattcggcctgtgtttgacccgagcaaggtgcgggccagtggaccgggcctggagcgcggcaaggtcggtgaggcagccaccttcactgtggactgctcagaggcaggcgaggcggagctgaccattgagatcctgtcggatgccggggtcaaggccgaggtgctgatccacaacaacgcggatggcacctaccacatcacctacagccctgccttccctggcacctacaccattaccatcaagtatggcgggcatcccgtgcccaaattccccacccgtgtccatgtgcagcctgcggtcgataccagtggcgtcaaggtctcagggcctggtgttgagccacacggtgtcctgcgggaggtgaccactgagttcactgtggatgcaagatccctaacagccacaggcggcaaccacgtgacggctcgtgtgctcaacccctcgggggccaagacagacacctatgtgacagacaatggggacggcacctaccgagtgcagtacaccgcctacgaggagggcgtgcatctggtggaggtcctgtatgatgaggtcgctgtgcccaagagccccttccgagtgggcgtgaccgagggctgtgatcccacccgcgtccgagccttcgggccaggcctggagggtggcttggtcaacaaggccaaccgattcactgtggagaccaggggagcgggcaccgggggccttggcctagccatcgagggtccctcggaagccaagatgtcctgcaaggacaacaaggatggtagctgcaccgtggagtacatccccttcactcctggagactatgacgtcaacatcaccttcggggggcggcccatcccagggagcccgttccgcgtgccagtgaaggatgtggtggaccctgggaaggtgaagtgctcagggccagggctgggggctggtgtcagggcccgggttcctcagaccttcacagtggattgcagtcaagctggccgggcgcccctgcaggtggctgtgctgggccccacaggtgtggccgagcctgtggaggtgcgggacaatggagatggcacccacactgtccactacaccccagccactgacgggccctacacggtagccgtcaagtatgctgaccaggaggtgccacgcagccccttcaagatcaaggtcctcccagctcatgatgccagcaaggtgcgggccagcggcccaggcctcaacgcctctggcatccctgccagcctgcctgtggagttcaccatcgacgcacgggacgcgggcgaggggttgctcactgtccagatcttggaccccgagggtaagcccaagaaggccaacatccgggacaatggggatggcacgtacactgtgtcctacctgccggacatgagtggccggtacaccatcaccatcaagtatggcggtgatgagatcccctactcgcccttccgcatccatgctctgcccactggggatgccagcaagtgcctcgtcacagtgtccattggaggccatggcctgggtgcctgcctgggccctcgaatccagattgggcaggagacggtgatcacggtggatgccaaggcagccggtgaggggaaggtgacatgcacggtgtccacgccggatggggcagagctcgatgtggatgtggttgagaaccatgacggtacctttgacatctactacacagcgcccgagccgggcaagtacgtcatcaccatccgcttcgggggtgagcacatccccaacagccccttccacgtgctggccacagaggagccagtggtgcctgtggagccaatggagtccatgctgaggcccttcaacctggtcatccccttcgcggtgcagaaaggggagctcacaggagaggtgcggatgccctcggggaagacggcacggcccaacatcaccgacaacaaggacggcaccatcacggtgaggtatgcacccactgagaaaggcctgcaccagatggggatcaagtatgacggcaaccacatccctgggagccccttacagttctatgtggatgccatcaacagccgccatgtcagtgcctatgggccaggcctgagccatggcatggtcaacaagccagccaccttcactattgtcaccaaagatgctggagaagggggtctgtcactggccgtggagggcccatccaaggcagagatcacctgtaaggacaacaaggatggcacctgcaccgtgtcctatctgccgactgcgcctggagactacagcatcatcgtgcgcttcgatgacaagcacatcccggggagccccttcacagccaagatcacaggtgatgactccatgaggacctcacagctgaatgtgggcacctccacggacgtgtcactgaagatcaccgagagtgatctgagccagctgaccgccagcatccgtgccccctcgggcaacgaggagccctgcctgctgaagcgcctgcccaaccggcacattgggatctccttcacccccaaggaggtcggggagcacgtggtgagcgtgcgcaagagtggcaagcatgtcaccaacagccccttcaagatcctggtggggccatctgagatcggggacgccagcaaggtgcgggtctggggcaaggggctttccgagggacacacattccaggtggcagagttcatcgtggacactcgcaatgcaggttatgggggcttggggctgagtattgaaggcccaagcaaggtggacatcaactgtgaggacatggaggacgggacatgcaaagtcacctactgccccaccgagcccggcacctacatcatcaacatcaagtttgctgacaagcacgtgcctggaagccccttcactgtgaaggtgaccggcgagggccgcatgaaggagagcatcacccggcggagacaggcaccttccatcgccaccatcggcagcacctgtgacctcaacctcaagatcccaggaaactggttccagatggtgtctgcccaggagcgcctgacacgcaccttcacacgcagcagccacacctacacccgcacggagcgcacggagatcagcaagacgcggggcggggagacaaagcgcgaggtgcgggtggaggagtccacccaggtcggcggggaccccttccctgctgtgtttggggacttcctgggccgggagcgcctgggatccttcggcagcatcacccggcagcaggagggtgaggccagctctcaggacatgactgcacaggtgaccagcccatcgggcaaggtggaagccgcagagatcgtcgagggcgaggacagcgcctacagcgtgcgctttgtgccccaggaaatggggccccatacggtcgctgtcaagtaccgtggccagcacgtgcccggcagcccctttcagttcactgtggggccgctgggtgaaggtggtgcccacaaggtgcgggccggaggcacagggctggagcgaggtgtggccggcgtgccagccgagttcagcatctggacccgggaggctggcgctgggggcctgtccattgctgtggagggtcctagcaaagcggagattgcatttgaggatcgcaaagatggctcctgcggcgtctcctatgtcgtccaggaaccaggtgactatgaggtctccatcaagttcaatgatgagcacatcccagacagcccctttgtggtgcctgtggcctccctctcggatgacgctcgccgtctcactgtcaccagcctccaggagacggggctcaaggtgaaccagccagcgtcctttgccgtgcagctgaacggtgcccggggcgtgattgatgcccgggtgcacacaccctcgggggctgtggaggagtgctacgtctctgagctggacagtgacaagcacaccatccgcttcatcccccacgagaatggcgtccactccatcgatgtcaagttcaacggtgcccacatccctggaagtcccttcaagatccgcgttggggagcagagccaggctggggacccaggcttggtgtcagcctacggtcctgggctcgagggaggcactaccggtgtgtcatcagagttcatcgtgaacaccctgaatgccggctcgggggccttgtctgtcaccattgatggcccctccaaggtgcagctggactgtcgggagtgtcctgagggccatgtggtcacttatactcccatggcccctggcaactacctcattgccatcaagtacggtggcccccagcacatcgtgggcagccccttcaaggccaaggtcactggtccgaggctgtccggaggccacagccttcacgaaacatccacggttctggtggagactgtgaccaagtcctcctcaagccggggctccagctacagctccatccccaagttctcctcagatgccagcaaggtggtgactcggggccctgggctgtcccaggccttcgtgggccagaagaactccttcaccgtggactgcagcaaagcaggcaccaacatgatgatggtgggcgtgcacggccccaagaccccctgtgaggaggtgtacgtgaagcacatggggaaccgggtgtacaatgtcacctacactgtcaaggagaaaggggactacatcctcattgtcaagtggggtgacgaaagtgtccctggaagccccttcaaagtcaaggtcccttgaatcccaaaagtgcctccccagcctcagcccccacctccagccacacacacattacacacacacacacacacacacaaatgtgccacacccagacacgcacagaatcagacactacaaacacctgccttgggggtgaagtgaaggcccagcctccccaccccaccgcgccccaggggttggaggaccttgtctgtgtcaggacagtgtccctccctgggaatgtgacatgagggccgactggggccaggctcaggggcagaggctgggacacaaggggctggcgagggctgcgaggccagggaagccctgagtttctggcggggctgagcagtgggggagcattgtgttgtgggtgtctgtgtgtgaggtcaccctcaaactgcaccgccggccagataccctcctgaccccgaggacttggtctggtctctctggtggctacaaccccagagttttaaggacttggaaaggaaagcacaatcagagaagaaaacagcccccgaaccagcaggagtggcctggcacatggaccggcctgagcgatgtgcactccacccaagccaggctcccagggggcctgatttctctctcactgtctctttttttaaaatggttgcacggctctgccccatggggggccttttttacacactgcgaggcccagctttctaggggacttttgcacatgtcatgcagctcagctgggagctgcttaggtggaaaactccaaataaagtgcggctgtcgca

SEQ ID NO: 20 - protein, 2692 aa, synthetic construct
  REGION 1..2692: note = FLNC amino acid sequence
MMNNSGYSDAGLGLGDETDEMPSTEKDLAEDAPWKKIQQNTFTRWCNEHLKCVGKRLTDLQRDLSDGLRLIALLEVLSQKRMYRKFHPRPNFRQMKLENVSVALEFLEREHIKLVSIDSKAIVDGNLKLILGLIWTLILHYSISMPMWEDEDDEDARKQTPKQRLLGWIQNKVPQLPITNFNRDWQDGKALGALVDNCAPGLCPDWEAWDPNQPVENAREAMQQADDWLGVPQVIAPEEIVDPNVDEHSVMTYLSQFPKAKLKPGAPVRSKQLNPKKAIAYGPGIEPQGNTVLQPAHFTVQTVDAGVGEVLVYIEDPEGHTEEAKVVPNNDKDRTYAVSYVPKVAGLHKVTVLFAGQNIERSPFEVNVGMALGDANKVSARGPGLEPVGNVANKPTYFDIYTAGAGTGDVAVVIVDPQGRRDTVEVALEDKGDSTFRCTYRPAMEGPHTVHVAFAGAPITRSPFPVHVSEACNPNACRASGRGLQPKGVRVKEVADFKVFTKGAGSGELKVTVKGPKGTEEPVKVREAGDGVFECEYYPVVPGKYVVTITWGGYAIPRSPFEVQVSPEAGVQKVRAWGPGLETGQVGKSADFVVEAIGTEVGTLGFSIEGPSQAKIECDDKGDGSCDVRYWPTEPGEYAVHVICDDEDIRDSPFIAHILPAPPDCFPDKVKAFGPGLEPTGCIVDKPAEFTIDARAAGKGDLKLYAQDADGCPIDIKVIPNGDGTFRCSYVPTKPIKHTIIISWGGVNVPKSPFRVNVGEGSHPERVKVYGPGVEKTGLKANEPTYFTVDCSEAGQGDVSIGIKCAPGVVGPAEADIDFDIIKNDNDTFTVKYTPPGAGRYTIMVLFANQEIPASPFHIKVDPSHDASKVKAEGPGLNRTGVEVGKPTHFTVLTKGAGKAKLDVQFAGTAKGEVVRDFEIIDNHDYSYTVKYTAVQQGNMAVTVTYGGDPVPKSPFVVNVAPPLDLSKIKVQGLNSKVAVGQEQAFSVNTRGAGGQGQLDVRMTSPSRRPIPCKLEPGGGAEAQAVRYMPPEEGPYKVDITYDGHPVPGSPFAVEGVLPPDPSKVCAYGPGLKGGLVGTPAPFSIDTKGAGTGGLGLTVEGPCEAKIECQDNGDGSCAVSYLPTEPGEYTINILFAEAHIPGSPFKATIRPVFDPSKVRASGPGLERGKVGEAATFTVDCSEAGEAELTIEILSDAGVKAEVLIHNNADGTYHITYSPAFPGTYTITIKYGGHPVPKFPTRVHVQPAVDTSGVKVSGPGVEPHGVLREVTTEFTVDARSLTATGGNHVTARVLNPSGAKTDTYVTDNGDGTYRVQYTAYEEGVHLVEVLYDEVAVPKSPFRVGVTEGCDPTRVRAFGPGLEGGLVNKANRFTVETRGAGTGGLGLAIEGPSEAKMSCKDNKDGSCTVEYIPFTPGDYDVNITFGGRPIPGSPFRVPVKDVVDPGKVKCSGPGLGAGVRARVPQTFTVDCSQAGRAPLQVAVLGPTGVAEPVEVRDNGDGTHTVHYTPATDGPYTVAVKYADQEVPRSPFKIKVLPAHDASKVRASGPGLNASGIPASLPVEFTIDARDAGEGLLTVQILDPEGKPKKANIRDNGDGTYTVSYLPDMSGRYTITIKYGGDEIPYSPFRIHALPTGDASKCLVTVSIGGHGLGACLGPRIQIGQETVITVDAKAAGEGKVTCTVSTPDGAELDVDVVENHDGTFDIYYTAPEPGKYVITIRFGGEHIPNSPFHVLATEEPVVPVEPMESMLRPFNLVIPFAVQKGELTGEVRMPSGKTARPNITDNKDGTITVRYAPTEKGLHQMGIKYDGNHIPGSPLQFYVDAINSRHVSAYGPGLSHGMVNKPATFTIVTKDAGEGGLSLAVEGPSKAEITCKDNKDGTCTVSYLPTAPGDYSIIVRFDDKHIPGSPFTAKITGDDSMRTSQLNVGTSTDVSLKITESDLSQLTASIRAPSGNEEPCLLKRLPNRHIGISFTPKEVGEHVVSVRKSGKHVTNSPFKILVGPSEIGDASKVRVWGKGLSEGHTFQVAEFIVDTRNAGYGGLGLSIEGPSKVDINCEDMEDGTCKVTYCPTEPGTYIINIKFADKHVPGSPFTVKVTGEGRMKESITRRRQAPSIATIGSTCDLNLKIPGNWFQMVSAQERLTRTFTRSSHTYTRTERTEISKTRGGETKREVRVEESTQVGGDPFPAVFGDFLGRERLGSFGSITRQQEGEASSQDMTAQVTSPSGKVEAAEIVEGEDSAYSVRFVPQEMGPHTVAVKYRGQHVPGSPFQFTVGPLGEGGAHKVRAGGTGLERGVAGVPAEFSIWTREAGAGGLSIAVEGPSKAEIAFEDRKDGSCGVSYVVQEPGDYEVSIKFNDEHIPDSPFVVPVASLSDDARRLTVTSLQETGLKVNQPASFAVQLNGARGVIDARVHTPSGAVEECYVSELDSDKHTIRFIPHENGVHSIDVKFNGAHIPGSPFKIRVGEQSQAGDPGLVSAYGPGLEGGTTGVSSEFIVNTLNAGSGALSVTIDGPSKVQLDCRECPEGHVVTYTPMAPGNYLIAIKYGGPQHIVGSPFKAKVTGPRLSGGHSLHETSTVLVETVTKSSSSRGSSYSSIPKFSSDASKVVTRGPGLSQAFVGQKNSFTVDCSKAGTNMMMVGVHGPKTPCEEVYVKHMGNRVYNVTYTVKEKGDYILIVKWGDESVPGSPFKVKVP

SEQ ID NO: 21 - DNA, 2762 bp, synthetic construct
  misc_feature 1..2762: note = HSP70 DNA sequence
  source 1..2762: mol_type = other DNA
gttggtctccatggcgatgctgaccgcctgcgcttcagcgcctaattgacaggaagggtcggcgctgggcttttaattgtgggggcttgctgggcctgcgcagttgtgggcagggggctggtgcggggagcgggttacgcgcgtccaggggagggtgggggcgtttgtgttgacaccgcccggccacgttacaatggagatactgtctctgacctcatgggccagtttggaaaaagattactgagttggagccgtctcaaatttgcagggagggacggggtggggggtggggggaccccggttgtgcagtttgatattgagggagcccccacctactcgctggggctgcgtaatctggacgtttccaaactgaagcgaaggcgtcgggagactaggcctcagagaaccatggctactgccaagggaatcgccataggcatcgacctgggcaccacctactcctgtgtgggggtgttccagcacggcaaggtggagatcatcgccaacgaccagggcaaccgcaccacccccagctacgtggccttcacagacaccgagcggctcattggggatgcggccaagaaccaggtagcaatgaatccccagaacactgtttttgatgctaaacgtctgatcggcaggaaatttaatgatcctgttgtacaagcagatatgaaactttggccttttcaagtgattaatgaaggaggcaagcccaaagtccttgtgtcctacaaaggggagaataaagctttctaccctgaggaaatctcttcgatggtattgactaagttgaaggagactgctgaggcctttttgggccaccctgtcaccaatgcagtgattaccgtgccagcctatttcaatgactctcaacgtcaggctactaaggatgcaggtgtgattgctggacttaatgtgctaagaatcatcaatgagcccacggctgctgccattgcctatggtttagataaaggaggtcaaggagaacgacatgtcctgatttttgatctgggtggaggcacatttgatgtgtcaattctgaccatagatgatgggatttttgaggtaaaggccactgctggggacactcacctgggtggggaggactttgacaacaggcttgtgagccacttcgtggaggagttcaagaggaaacacaaaaaggacatcagccagaacaagcgagccgtgaggcggctgcgcaccgcctgcgagagggccaagaggaccctgtcgtccagcacccaggccaacctagaaattgattcactttatgaaggcattgacttctatacatccatcaccagagctcgatttgaagagttgtgtgcagacctgtttaggggtaccctggagcctgtagaaaaagcgcttcgggatgccaagatggataaggctaaaatccatgacattgttttagtagggggctccacccgcatccccaaggtgcagcggctgcttcaggactacttcaatggacgtgatctcaacaagagcatcaaccctgatgaggccgtagcatatggggctgcggtacaagcagccatcctgatgggggacaagtctgagaaggtacaggacctgctgctgctggacgtggctcccctgtccctggggctggagacggctgggggcgtgatgactgccctgataaagcgcaactccaccatccccaccaagcagacacagattttcaccacctactctgacaaccaacccggggtgctgatccaggtgtatgagggcgagagggccatgacaaaggacaacaacctgctggggcggtttgacctgactggaatccctccagcacccaggggagttcctcagatcgaggtgacgtttgacattgatgccaatggtattctcaatgtcacagccacggacaagagcaccggcaaggtgaacaagatcaccatcaccaatgacaagggccgcctgagcaaggaggagattgagcgcatggttctggatgctgagaaatataaagctgaagatgaggtccagagggagaaaattgctgcaaagaatgccttagaatcctatgcttttaacatgaagagtgttgtgagtgatgaaggtttgaagggcaagattagtgagtctgataaaaataaaatattggataaatgcaacgagctcctttcgtggctggaggtcaatcaactggcagagaaagatgagtttgatcataagagaaaggaattggagcagatgtgtaaccctatcatcacaaaactctaccaaggaggatgcactgggcctgcctgcggaacagggtatgtgcctggaaggcctgccacaggccccacaattgaagaagtagattaattctttttagaactgaagcatcctaggatgcctctacatgtatttcattcccctcatcttcaaacatcattattattcttgaccagacctgaatctaagttaccatcccttggaaattctggagaaggagtctcatgcaccacctatcacactccctcacatcctgtttctgactttggaatggactcaggaaaactaggcccctctttaaaccgtgtgatgtatttgaatgtctgttatttccagccaccctaacattcttcttcctgtgtggatgcttatttgtcaatcagtaaatttgttcgtaaagaaaattacttctggtatttaggctgtgaatgtaccttgaaggggagagttcatggagagagcatgtgttctctgattgtgaggtcactgtgaatgattaaattggtaagggtaaagta

SEQ ID NO: 22 - protein, 641 aa, synthetic construct
  REGION 1..641: note = HSP70 amino acid sequence
MATAKGIAIGIDLGTTYSCVGVFQHGKVEIIANDQGNRTTPSYVAFTDTERLIGDAAKNQVAMNPQNTVFDAKRLIGRKFNDPVVQADMKLWPFQVINEGGKPKVLVSYKGENKAFYPEEISSMVLTKLKETAEAFLGHPVTNAVITVPAYFNDSQRQATKDAGVIAGLNVLRIINEPTAAAIAYGLDKGGQGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKALRDAKMDKAKIHDIVLVGGSTRIPKVQRLLQDYFNGRDLNKSINPDEAVAYGAAVQAAILMGDKSEKVQDLLLLDVAPLSLGLETAGGVMTALIKRNSTIPTKQTQIFTTYSDNQPGVLIQVYEGERAMTKDNNLLGRFDLTGIPPAPRGVPQIEVTFDIDANGILNVTATDKSTGKVNKITITNDKGRLSKEEIERMVLDAEKYKAEDEVQREKIAAKNALESYAFNMKSVVSDEGLKGKISESDKNKILDKCNELLSWLEVNQLAEKDEFDHKRKELEQMCNPIITKLYQGGCTGPACGTGYVPGRPATGPTIEEVD

SEQ ID NO: 23 - DNA, 3231 bp, synthetic construct
  misc_feature 1..3231: note = MAN2B1 DNA sequence
  source 1..3231: mol_type = other DNA
cccggccgggtctgggggcggggcgtttgcccggcctttccagggccggggaaccccaggaggaagctgctgagccatgggcgcctacgcgcgggcttcgggggtctgcgctcgcggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttaggtctgctgggatgggccctccaagcccaagcctcctgctccgggggcagaccagactctgactctcctcttggggctgctgccattaaaacgctactactaagactcaggtcaaaaaaa

SEQ ID NO: 24 - protein, 1011 aa, synthetic construct
  REGION 1..1011: note = MAN2B1 amino acid sequence
MGAYARASGVCARGCLDSAGPWTMSRALRPPLPPLCFFLLLLAAAGARAGGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG

(19) United States
(12) Patent Application Publication
(10) Pub. No.: US 2026/0258495 A1
(43) Pub. Date: Sep. 3, 2026

(54) BIOMARKER FOR EARLY DIAGNOSIS OF ALZHEIMER'S DISEASE AND USE THEREOF

(71) Applicant: DAEGU GYEONGBUK INSTITUTE OF SCIENCE AND TECHNOLOGY, Daegu (KR)

(72) Inventors: Jong Hyuk YOON, Daegu (KR); Yeon Suk JO, Daegu (KR); Geun A PARK, Daegu (KR); Sung Won BAE, Daegu (KR); Yang Woo KWON, Daegu (KR); Seul Ah LEE, Daegu (KR); Kuk In JANG, Daegu (KR); Chan Y LEE, Daegu (KR); Yong Seok OH, Daegu (KR); Ha Gyeong LEE, Daegu (KR); Da Young KWON, Daegu (KR)

(21) Appl. No.: 18/993,162

(22) PCT Filed: Jul. 11, 2023
(86) PCT No.: PCT/KR2023/009845

§ 371 Date: Jan. 10, 2025

(30) Foreign Application Priority Data
Jul. 12, 2022 (KR) 10-2022-0085784
Jul. 11, 2023 (KR) 10-2023-0089693

(51) Int. Cl. C12Q 1/6876

(52) U.S. Cl. CPC C12Q 1/6876; C12Q 2600/118; C12Q 2600/158; G01N 2800/2821; G01N 2800/50; G01N 2800/60

(57) ABSTRACT

The present invention relates to a biomarker for the early diagnosis of Alzheimer's disease and a use thereof, and more particularly, the present invention relates to: a biomarker composition for the early diagnosis of Alzheimer's disease, the biomarker composition comprising one or more genes selected from the group consisting of alpha-2-macroglobulin (A2M), creatine kinase M-type (CKM), filamin-A (FLNA), integral alpha-IIb (ITGA2B), alpha-1-acid glycoprotein 2 (ORM2), phospholipid transfer protein (PLTP), haptoglobin (HP), sulfhydryl oxidase 1 (QSOX1), protein-glutamine gamma-glutamyltransferase 2 (TGM2), filamin C (FLNC), heat shock protein 70 (HSP70) and lysomal alpha-mannosidase (MAN2B1), or a protein expressed from the genes; a method for the early diagnosis of Alzheimer's disease; a diagnostic kit for the early diagnosis of Alzheimer's disease; and a method for providing information for predicting and diagnosing Alzheimer's disease.

### TECHNICAL FIELD

The present disclosure relates to a biomarker for the early diagnosis of Alzheimer's disease and use thereof.

### BACKGROUND ART

Alzheimer's disease (AD) is characterized by dementia and the loss of cognitive abilities, including reasoning and memory. Currently, treatments for Alzheimer's disease involve the use of acetylcholinesterase (ACE) inhibitors and N-methyl-D-aspartate (NMDA) receptor antagonists. While these drugs target cognitive impairments and are effective in the early stages of Alzheimer's disease, their therapeutic and improvement effects in later stages remain controversial. In cases where such drugs are administered at advanced stages of the disease, only trivial therapeutic effects are observed.

Existing diagnostic methods for Alzheimer's disease include clinical mental state examinations (MSE) and brain imaging techniques, such as amyloid positron emission tomography (PET). However, these methods face limitations in diagnosing early-stage Alzheimer's disease. Furthermore, the progression of Alzheimer's disease involves numerous molecular mechanisms, many of which are not yet fully understood. Therefore, there is a pressing need for the development of technologies that enable accurate diagnosis at early stages of Alzheimer's disease.

Additionally, diagnostic samples for Alzheimer's disease typically include blood or cerebrospinal fluid (CSF) from patients. When using blood samples, compared to CSF, the diagnostic process offers cost savings and shorter analysis times. Consequently, recent efforts have focused on identifying blood-based biomarkers for Alzheimer's disease. However, most identified biomarkers have failed cross-validation attempts, underscoring the need for discovering new, reliable biomarkers.

### DISCLOSURE OF INVENTION

### Technical Problem

Leading to the present disclosure, intensive and thorough research conducted by the present inventors to identify reliable and accurate blood-based biomarkers for the early diagnosis of Alzheimer's disease address resulted in discovering novel biomarkers that exhibit significant differences in expression levels between normal individuals and early Alzheimer's disease patients, derived from extracellular vesicles in plasma and verifying the diagnostic reliability of these biomarkers using plasma samples from actual Alzheimer's disease patients.

Accordingly, the present disclosure aims to provide a biomarker composition for early Alzheimer's disease diagnosis, the composition including at least one gene selected from the group consisting of A2M (alpha-2-macroglobulin), CKM (creatine kinase M-type), FLNA (filamin-A), ITGA2B (integrin alpha-IIb), ORM2 (alpha-1-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase), or a protein expressed therefrom.

Also, the present disclosure is to provide a composition for early Alzheimer's disease diagnosis, the composition including a substance for measuring an mRNA or protein expression level of at least one gene selected from the group consisting of A2M (alpha-2-macroglobulin), CKM (creatine kinase M-type), FLNA (filamin-A), ITGA2B (integrin alpha-IIb), ORM2 (alpha-i-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase).

Furthermore, the present disclosure is to provide an early Alzheimer's disease diagnostic kit including the early Alzheimer's disease diagnostic composition.

Moreover, the present disclosure is to provide a method for providing information for predicting and diagnosing Alzheimer's disease, the method including the steps of: (a) measuring an mRNA level or protein expression level of at least one selected from the group consisting of A2M (alpha-2-macroglobulin), CKM (creatine kinase M-type), FLNA (filamin-A), ITGA2B (integrin alpha-IIb), ORM2 (alpha-i-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase) from a biological sample isolated from a subject suspected of having Alzheimer's disease; and (b) comparing the measurement result of step (a) with an mRNA or protein expression level of the same gene in a control sample.

### Solution to Problem

To achieve the goals, the present disclosure provides a biomarker composition for early Alzheimer's disease diagnosis, the composition including at least one gene selected from the group consisting of A2M (alpha-2-macroglobulin), CKM (creatine kinase M-type), FLNA (filamin-A), ITGA2B (integrin alpha-IIb), ORM2 (alpha-1-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase), or a protein expressed therefrom.

In an embodiment of the present disclosure, the gene or the protein may have an increased expression level during the onset of Alzheimer's disease compared to normal individuals.

In an embodiment of the present disclosure, the biomarker composition for early Alzheimer's disease diagnosis may include four to six different types of the aforementioned genes or proteins expressed therefrom.

Additionally, the present disclosure provides a composition for early Alzheimer's disease diagnosis, the composition including a substance for measuring an mRNA or protein level of at least one gene selected from the group consisting of A2M, CKM, FLNA, ITGA2B, ORM2, PLTP, HP, QSOX1, TGM2, FLNC, HSP70, and MAN2B1.

In an embodiment of the present disclosure, the composition for early Alzheimer's disease diagnosis may include a substance for measuring mRNA or protein levels of four to six different types of the aforementioned genes.

In an embodiment of the present disclosure, the substance may include a primer, a probe, or an antibody that specifically binds to the gene or protein.

Furthermore, the present disclosure provides a diagnostic kit for early Alzheimer's disease, the kit including the composition for early Alzheimer's disease diagnosis according to the present disclosure.

Additionally, the present disclosure provides a method for providing information for predicting and diagnosing Alzheimer's disease, the method including the steps of: (a) measuring an mRNA level or protein expression level of at least one selected from the group consisting of A2M, CKM, FLNA, ITGA2B, ORM2, PLTP, HP, QSOX1, TGM2, FLNC, HSP70, and MAN2B1 from a biological sample isolated from a subject suspected of having Alzheimer's disease; and (b) comparing the measurement result of step (a) with an mRNA or protein expression level of the same gene in a normal control sample.

In an embodiment of the present disclosure, the biological sample may be blood or plasma.

In an embodiment of the present disclosure, the sample may be extracellular vesicles derived from plasma.

In an embodiment of the present disclosure, when the mRNA or protein expression level of the gene is increased compared to a normal control group, the subject may be determined to be at an early stage of Alzheimer's disease.

In an embodiment of the present disclosure, the mRNA levels or protein expression levels of A2M, CKM, FLNA, ITGA2B, ORM2, and PLTP may increase during early-stage Alzheimer's disease compared to a normal control group and subsequently decrease during late-stage Alzheimer's disease.

In an embodiment of the present disclosure, the mRNA levels or protein expression levels of HP, QSOX1, and TGM2 may increase only during early-stage Alzheimer's disease.

In an embodiment of the present disclosure, the mRNA levels or protein expression levels of FLNC, HSP70, and MAN2B1 may increase during both early and late stages of Alzheimer's disease.

### Advantageous Effects of Invention

When applied, the biomarkers for early Alzheimer's disease diagnosis provided in the present disclosure allows for improving diagnostic accuracy by using extracellular vesicles, which can be easily obtained from plasma, as a sample, and enables the accurate, sensitive, and specific diagnosis of Alzheimer's disease, particularly during the early stages of disease progression.

### BRIEF DESCRIPTION OF DRAWINGS

FIG. 1 illustrates the multi-proteomic preparation process and confirmation of plasma-derived extracellular vesicles (EVs) from wild-type (WT) mice and 5xFAD mice: (a) shows the workflow of the biomarker discovery process for Alzheimer's disease diagnosis according to the present disclosure; (b) displays immunohistochemistry images of the medial prefrontal cortex (mPFC) and hippocampus (HPC) of WT and 5xFAD mice using anti-AB1-16 (clone 6E10); (c) presents a graphical comparison of the immunostaining intensities shown in (b); (d) shows the results of Western blot analysis of amyloid precursor protein (APP) levels in brain tissue lysates from WT and 5xFAD mice; (e) provides a quantitative graph of APP protein expression levels from (d); (f) depicts the size analysis of plasma-derived EVs using NanoSight LM10; (g) illustrates Western blot results confirming plasma-derived EV marker proteins (anti-CD9, anti-CD63, anti-CD81).

FIG. 2 illustrates proteomic analysis results of the cerebral cortex, hippocampus, and plasma-derived extracellular vesicles from 3-month-old and 6-month-old WT and 5xFAD mice: (a) presents a Venn diagram of the identified proteins; and (b) shows gene ontology (GO)-based functional annotations for the hippocampus, cortex, and plasma-derived EVs from 3-month-old WT and 5xFAD mice, where “BP,” “CC,” and “MF” represent biological process, cellular component, and molecular function, respectively.

FIG. 3 illustrates GO-based functional annotations for proteomic differences between 3-month-old and 6-month-old Alzheimer-like 5xFAD mice.

FIG. 4 shows Western blot results for selected Alzheimer's disease biomarker candidates in plasma-derived extracellular vesicles (a), cerebral cortex (b), and hippocampus (c) from 3-month-old WT and 5xFAD mice.

FIG. 5 presents Western blot results for plasma-derived extracellular vesicle markers using antibodies from plasma samples obtained at different stages of Alzheimer's disease progression.

FIG. 6 demonstrates the diagnostic utility of the biomarkers identified in the present disclosure using plasma-derived EV samples from normal, early-stage, and late-stage Alzheimer's disease patients: (a) summarizes Western blot results in a table format, where: Class 1 indicates proteins upregulated only in early-stage Alzheimer's disease and downregulated in late-stage Alzheimer's disease compared to normal, Class 2 represents proteins upregulated exclusively in early-stage Alzheimer's disease, and Class 3 denotes proteins upregulated in both early- and late-stage Alzheimer's disease; (b) to (d) depict scatterplots showing expression levels of Class 1, Class 2, and Class 3 markers, respectively.

FIG. 7 shows Western blot results indicating changes in protein expression levels of markers classified under Class 1 in plasma-derived EV samples from normal, early-stage, and late-stage Alzheimer's disease patients.

FIG. 8 illustrates Western blot results showing changes in protein expression levels of Class 2 markers in plasma-derived EV samples from normal, early-stage, and late-stage Alzheimer's disease patients.

FIG. 9 demonstrates Western blot results for changes in protein expression levels of Class 3 markers in plasma-derived EV samples from normal, early-stage, and late-stage Alzheimer's disease patients.

FIG. 10 provides scatterplots of protein expression levels for PF4 and TLN1, candidate biomarkers identified in the present disclosure, in plasma-derived EV samples from normal, early-stage, and late-stage Alzheimer's disease patients to assess their diagnostic utility.

FIG. 11 shows graphs and AUC-ROC curves for analysis results of the diagnostic performance of the early Alzheimer's disease biomarkers identified in the present disclosure using machine learning, illustrating diagnostic accuracy, sensitivity, and specificity based on the number of biomarker combinations for normal vs. early-stage AD groups (a), normal vs. late-stage AD groups (b), and early-stage vs. late-stage AD groups (c).

### BEST MODE FOR CARRYING OUT THE INVENTION

The present disclosure identifies novel biomarkers that enable rapid and accurate early diagnosis of Alzheimer's disease, characterized by their ability to diagnose early-stage Alzheimer's disease effectively.

In the course of research to discover biomarkers capable of diagnosing and predicting early-stage Alzheimer's disease with precision and efficacy, the present inventors identified genes showing significant differences in expression levels in plasma-derived extracellular vesicles from early-stage Alzheimer's disease patients compared to normal individuals. These identified markers were confirmed to be applicable as biomarkers for early Alzheimer's disease diagnosis.

In an embodiment of the present disclosure, classification was made of wild-type (WT), early-stage Alzheimer's disease, and late-stage Alzheimer's disease mouse groups to identify diagnostic markers for Alzheimer's disease. Plasma-derived extracellular vesicles were then isolated from each mouse group, and proteins exhibiting differences in expression levels between groups were identified. The results revealed that the proteins listed in [Table 2] of Example 2 exhibited differential expression.

Furthermore, to verify whether the candidate markers identified in [Table 2] from Alzheimer's disease-induced mice could be useful for diagnosing the onset of Alzheimer's disease in humans, plasma-derived extracellular vesicles were isolated from the blood of early- and late-stage Alzheimer's disease patients as defined by their Mini-Mental State Examination (MMSE) scores. Subsequently, the expression levels of the identified genes in extracellular vesicle samples from Alzheimer's disease patients were analyzed. Proteins showing significant differences in expression levels were evaluated in EVs from normal, early-stage Alzheimer's disease, and late-stage Alzheimer's disease groups.

The analysis identified 12 proteins, including A2M (alpha-2-macroglobulin), CKM (creatine kinase M-type), FLNA (filamin-A), ITGA2B (integrin alpha-IIb), ORM2 (alpha-i-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase), which were significantly upregulated in samples from patients diagnosed with early-stage Alzheimer's disease compared to the normal group.

In addition, analysis of Alzheimer's disease patient samples further showed that the mRNA or protein expression levels of A2M(alpha-2-macroglobulin), CKM(creatine kinase M-type), FLNA(filamin-A), ITGA2B(Integrin alpha-IIb), ORM2(alpha-i-acid glycoprotein 2), and PLTP(phospholipid transfer protein) genes were upregulated during early-stage Alzheimer's disease compared to the normal control group, but tended to decrease in the late-stage Alzheimer's disease group.

In contrast, the mRNA or protein expression levels of HP (haptoglobin), QSOX1(sulfhydryl oxidase 1), and TGM2 (protein-glutamine gamma-glutamyltransferase 2) genes were observed to increase exclusively during the early stage of Alzheimer's disease. Meanwhile, the mRNA or protein expression levels of FLNC (filamin C), HSP70(heat shock protein 70), and MAN2B1(lysosomal alpha-mannosidase) were upregulated during both early and late stages of the disease.

Thus, the inventors concluded that the identified markers could be utilized as biomarkers for diagnosing Alzheimer's disease.

Accordingly, the present disclosure provides a biomarker composition for early Alzheimer's disease diagnosis, the composition including at least one gene selected from the group consisting of A2M(alpha-2-macroglobulin), CKM(creatine kinase M-type), FLNA(filamin-A), ITGA2B(Integrin alpha-IIb), ORM2(alpha-1-acid glycoprotein 2), PLTP(phospholipid transfer protein), HP(haptoglobin), QSOX1(sulfhydryl oxidase 1), TGM2 (protein-glutamine gamma-glutamyltransferase 2), FLNC(filamin C), HSP70(heat shock protein 70), and MAN2B1 (lysosomal alpha-mannosidase), or a protein expressed from the gene.

Herein, A2M (alpha-2-macroglobulin) is represented by SEQ ID NO: 1 for nucleotide sequence and SEQ ID NO: 2 for amino acid sequence; CKM (creatine kinase M-type) by SEQ ID NO: 3 for nucleotide sequence and SEQ ID NO: 4 for amino acid sequence; FLNA (filamin-A) by SEQ ID NO: 5 for nucleotide sequence and SEQ ID NO: 6 for amino acid sequence; ITGA2B (Integrin alpha-IIb) by SEQ ID NO: 7 for nucleotide sequence and SEQ ID NO: 8 for amino acid sequence; and ORM2 (alpha-i-acid glycoprotein 2) by SEQ ID NO: 9 for nucleotide sequence and SEQ ID NO: 10 for amino acid sequence.

Also, representation is made of PLTP (phospholipid transfer protein) by SEQ ID NO: 11 for nucleotide sequence and SEQ ID NO: 12 for amino acid sequence; HP (haptoglobin) by SEQ ID NO: 13 for nucleotide sequence and SEQ ID NO: 14 for amino acid sequence; and QSOX1 (sulfhydryl oxidase 1) by SEQ ID NO: 15 for nucleotide sequence and SEQ ID NO: 16 for amino acid sequence.

Furthermore, representation is made of TGM2 (protein-glutamine gamma-glutamyltransferase 2) by SEQ ID NO: 17 for nucleotide sequence and SEQ ID NO: 18 for amino acid sequence; FLNC(filamin C) by SEQ ID NO: 19 for nucleotide sequence and SEQ ID NO: 20 for amino acid sequence; HSP70 (heat shock protein 70) by SEQ ID NO: 21 for nucleotide sequence and SEQ ID NO: 22 for amino acid sequence; and MAN2B1 (lysosomal alpha-mannosidase) by SEQ ID NO: 23 for nucleotide sequence and SEQ ID NO: 24 for amino acid sequence.

The biomarker composition of the present disclosure may include at least one of the identified marker genes or proteins expressed therefrom. The at least one marker gene or protein may consist of any one marker, a combination of two markers, three markers, four markers, five markers, six markers, seven markers, eight markers, nine markers, ten markers, 11 markers, or all selected from the 12 markers discovered in the present disclosure. Preferably, the composition includes four to six different marker genes or proteins expressed from the marker genes, with more preference for five different marker genes or proteins expressed from the marker genes.

Machine learning analysis was conducted with respect to the 12 marker genes identified in the present disclosure to make an optimal marker combination for diagnosing early Alzheimer's disease with the highest accuracy. As a result, the combination of five markers was detected to be optimal for diagnosing early Alzheimer's disease with the highest accuracy, sensitivity, and specificity. This combination proved superior to using fewer than four or more than six markers.

For the most effective early Alzheimer's disease diagnosis, it is preferable to use a combination of five markers from the identified 12. The most optimal combination comprises ITGA2B, FLNC, CKM, TGM2, and MAN2B1.

In an embodiment, machine learning analysis applied to the biomarkers identified in the present disclosure was conducted to make a combination able to distinguish normal and early Alzheimer's disease patients, resulting in the combination of ITGA2B, FLNC, CKM, TGM2, and MAN2B1 that was identified to diagnose early Alzheimer's disease patients with an accuracy of 78.5%.

Additionally, the present disclosure provides a diagnostic composition for early Alzheimer's disease, the composition including a substance for measuring an mRNA or protein level of at least one gene selected from the group consisting of A2M (alpha-2-macroglobulin), CKM (creatine kinase M-type), FLNA (filamin-A), ITGA2B (Integrin alpha-IIb), ORM2 (alpha-i-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase).

The substance may include a primer, a probe, or an antibody specifically binding to the gene or protein. In an embodiment, the target protein was detected using an antibody specific therefor.

As used herein, the term “biomarker” refers to an organic biomolecule such as a polypeptide or nucleic acid (e.g., mRNA), a lipid, a glycolipid, a glycoprotein, and a sugar (monosaccharide, disaccharide, oligosaccharide, etc.) that exhibit increased or decreased expression in tissues, cells, or blood upon the onset of Alzheimer's disease. Specifically, 12 genes or their protein products, which show increased expression levels in plasma-derived extracellular vesicles of individuals with Alzheimer's disease compared to normal individuals, as identified herein, may be used as biomarkers of the present disclosure.

The extracellular vesicles (EVs) described in the present disclosure are vesicles generated by cells and released extracellularly, including exosomes and microvesicles. EVs are known to mediate intercellular signaling and are involved in various phenomena such as cell differentiation, immune cell activation, secretion of inflammatory substances, tumor malignancy, and metastasis. These vesicles contain proteins and RNA derived from their originating cells, making them particularly useful for disease diagnosis. For example, EVs from disease-specific cells, such as cancer cells, carry disease-specific proteins and RNA, enabling their application in diagnostics.

As used herein, the term “gene expression level” preferably refers to the mRNA level expressed by the gene, that is, the quantity of mRNA. A substance capable of measuring the level may include a primer or a probe specific to the gene. The primer or probe specific for the gene can specifically amplify the entire gene or specific regions thereof and may be designed using methods well known in the art.

The term “primer”, as used herein, refers to a single-stranded oligonucleotide capable of acting as an initiation point for template-directed DNA synthesis under appropriate conditions (e.g., suitable temperatures and buffers in the presence of four nucleoside triphosphates and a polymerase enzyme). The suitable length of the primer may vary depending on factors such as temperature and the intended application. The primer sequence does not need to be perfectly complementary to the template sequence but must exhibit sufficient complementarity to hybridize with the template and function effectively. The sequence of the primer does not need to be completely complementary to a partial sequence of the template; it is sufficient to have sufficient complementarity to hybridize with the template and perform the original function of the primer. Preferably, the primers are suitable for use in gene amplification reactions.

Amplification reactions refer to processes that amplify nucleic acid molecules. Well-known gene amplification methods in the art include polymerase chain reaction (PCR), reverse transcription PCR (RT-PCR), ligase chain reaction (LCR), transcription-mediated amplification (TMA), and nucleic acid sequence-based amplification (NASBA), among others.

The term “probe”, as used herein, refers to a linear oligomer composed of natural or modified monomers or linkages, including deoxyribonucleotides and ribonucleotides. Probes can hybridize specifically to a target nucleotide sequence and may be naturally occurring or synthetically produced. The probe according to the present disclosure may be single-stranded and may be preferably an oligodeoxyribonucleotide. The probes of the present disclosure may include natural dNMPs (e.g., dAMP, dGMP, dCMP, and dTMP), nucleotide analogs, or derivatives. Additionally, probes may include ribonucleotides. For instance, the probes of the present disclosure may include backbone-modified nucleotides such as peptide nucleic acids (PNA) (M. Egholm et al., Nature, 365:566-568(1993)), phosphorothioate DNA, phosphorodithioate DNA, phosphoroamidate DNA, amide-linked DNA, MMI-linked DNA, 2′-O-methyl RNA, alpha-DNA, and methyl phosphonate DNA, sugar-modified nucleotides such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and base-modified nucleotides such as C-5 substituted pyrimidines (substituents including fluoro-, bromo-, chloro-, iodo-, methyl-, ethyl-, vinyl-, formyl-, etityl-, propinly, alkynyl-, thiazolyl-, imidazolyl-, pyridyl), C-7 substituted 7-deazapurines (substituents including luoro-, bromo-, chloro-, iodo-, methyl-, ethyl-, vinyl-, formyl-, alkynyl-, alkenyl-, thiazolyl-, imidazolyl-, pyridyl-), inocines, and diaminopuries.

Substances capable of measuring protein levels according to the present disclosure include antibodies, such as polyclonal, monoclonal, or recombinant antibodies, that specifically bind to the protein products expressed by the marker genes of the present disclosure.

As the “antibody”, any antibody that can be produced using known techniques in the art may be available. Polyclonal antibodies can be produced by injecting the antigenic protein into an animal and collecting the serum containing antibodies via methods widely known in the art. Such polyclonal antibodies can be produced in any host animals such as goats, rabbits, sheep, monkeys, horses, pigs, cows, and dogs. Monoclonal antibodies can be produced using widely known hybridoma techniques (Kohler et al., European Journal of Immunology, 6:511-519, 1976) or phage display libraries (Clackson et al., Nature, 352:624-628, 1991 and Marks et al, J. Mol. Biol., 222:58, 1-597, 1991). In addition, the antibodies of the present disclosure may include both full-length antibodies (two heavy and two light chains) and functional fragments thereof. The term “functional fragment” refers to a fragment that retains at least antigen-binding capability, as exemplified by Fab, F(ab′), F(ab′)₂, and Fv fragments.

Furthermore, the present disclosure provides a diagnostic kit for early-stage Alzheimer's disease, which includes the biomarker or diagnostic composition for early-stage Alzheimer's disease as described herein.

The diagnostic kit of the present disclosure may include primers, probes, or antibodies capable of measuring the expression level of the marker genes or the amount of protein expressed by these genes. The definitions of these components are as described in the foregoing.

If the diagnostic kit of the present disclosure is applied to a PCR amplification process, the kit may optionally include reagents necessary for PCR amplification, such as buffers, DNA polymerases (e.g., thermostable DNA polymerases derived from Thermus aquaticus (Taq), Thermus thermophilus (Tth), Thermus filiformis, Thermis flavus, Thermococcus literalis, or Pyrococcus furiosus (Pfu)), DNA polymerase cofactors, and dNTPs. If the diagnostic kit is applied to immunoassays, the kit may optionally include secondary antibodies and labeled substrates.

Additionally, the diagnostic kit of the present disclosure may include an instruction manual indicating that if the expression levels of the genes or proteins corresponding to the biomarkers are measured and found to be elevated compared to a normal control group, it may be determined that early-stage Alzheimer's disease has developed.

Moreover, the diagnostic kit according to the present disclosure can be configured with multiple separate packaging or compartments containing the aforementioned reagent components.

The present disclosure also provides a microarray for diagnosing early-stage Alzheimer's disease, which includes the biomarkers for diagnosing early-stage Alzheimer's disease.

In the microarray of the present disclosure, the primer, probe, or antibody capable of measuring the expression level of the marker protein or the gene coding therefor can serve as a hybridizable array element and is immobilized on a substrate. Suitable substrates include solid or semi-solid supports, such as membranes, filters, chips, slides, wafers, fibers, magnetic beads, non-magnetic beads, gels, tubes, plates, polymers, microparticles, and capillaries. The hybridizable array elements are arrayed and immobilized on the substrate using chemical or covalent methods such as UV treatment. For instance, the hybridizable array elements can be bound to a glass surface modified to contain epoxy compounds or aldehyde groups or can be UV-bonded on a polylysine-coated surface. Additionally, the hybridizable array elements may be linked to the substrate via linkers (e.g., ethylene glycol oligomers or diamines).

When the sample applied to the microarray is a nucleic acid, it may be labeled and hybridized with the array elements on the microarray. The hybridization conditions may vary, and the detection and analysis of hybridization can be conducted according to the labeling material used.

Furthermore, the present disclosure provides a method for predicting and diagnosing Alzheimer's disease, which includes the steps of: (a) measuring an mRNA level or protein expression level of at least one gene selected from the group consisting of A2M (alpha-2-macroglobulin), CKM (creatine kinase M-type), FLNA (filamin-A), ITGA2B (Integrin alpha-IIb), ORM2 (alpha-i-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase) in a biological sample obtained from a subject suspected of having Alzheimer's disease; and (b) comparing the results of step (a) with the mRNA or protein expression level of the corresponding gene measured in a control sample.

Measuring the expression levels of genes or the amount of proteins can be carried out using a process known in the art to isolate mRNA or protein from a biological sample.

In the present disclosure, the term “biological sample” refers to a sample collected from a subject in which the expression levels of the genes or proteins vary according to the onset or progression of Alzheimer's disease compared to a normal control. Examples of such samples include, but are not limited to, blood, serum, plasma, saliva, and urine, with preference for plasma.

The biomarkers identified in the present disclosure are particularly advantageous as they can be detected in extracellular vesicles derived from plasma. Unlike tissues or cerebrospinal fluid, which are difficult to obtain, blood can be more easily used for diagnostic purposes.

Measuring gene expression levels preferably involves assessing mRNA levels. Techniques for measuring mRNA levels include reverse transcription polymerase chain reaction (RT-PCR), real-time RT-PCR, RNase protection assays, Northern blotting, and DNA microarrays, but are not limited thereto.

Methods for measuring the amount or activity of proteins may include various techniques known in the art, such as Western blotting, Northern blotting, ELISA (enzyme-linked immunosorbent assay), radioimmunoassay (RIA), radial immunodiffusion, and immunoprecipitation assays, among others.

Protein levels can be measured using antibodies. In this regard, the marker proteins in the biological sample form conjugates with antibodies specific therefor, that is, antigen-antibody complexes. The amount of antigen-antibody complex formed can be quantified through the signal intensity of a detection label, which may be selected from the group consisting of enzymes, fluorescent substances, ligands, luminescent substances, microparticles, redox molecules, and radioisotopes, but with no limitations thereto. Analytical methods for protein level measurement include, but are not limited to, Western blotting, ELISA, radioimmunoassay, radial immunodiffusion, immunodiffusion, immunoprecipitation, tissue immunostaining, complement fixation assays, FACS, and protein microarrays.

Therefore, through these detection methods, the mRNA or protein expression levels of the marker genes in the control group can be compared with those in patients or suspected patients with Alzheimer's disease. By comparing the expression levels, it is possible to predict and diagnose the onset, progression stage, or prognosis of Alzheimer's disease.

More specifically, the method for predicting or diagnosing Alzheimer's disease allows for determining that Alzheimer's disease is induced if the expression level of the marker gene or protein is elevated compared to a normal control sample. Particularly, if the gene or protein expression level is confirmed to be elevated compared to the control group, it may indicate the early stage of Alzheimer's disease.

As described above, the novel biomarkers identified in the present disclosure enable the accurate and rapid prediction and diagnosis of Alzheimer's disease progression, especially at the early stage, by measuring the expression levels of these markers in plasma-derived extracellular vesicles.

### MODE FOR CARRYING OUT THE INVENTION

A better understanding of the present disclosure may be obtained through the following examples, which are set forth illustrate, but are not to be construed to limit, the present disclosure.

### <Preparatory Examples and Experimental Methods>

### Experimental Animals and Sample Preparation

All experimental procedures involving animals were approved by the Korea Brain Research Institute Animal Use and Care Committee. 5xFAD hemizygous mice (B6.Cg-Tg (APPSwFlLon, PSEN1*M146L*L286V) 6799Vas/Mmj ax), MMRRC stock #34848) and wild-type littermates thereof were produced by crossing with C57BL/6J (JAX stock #000664) females (Jackson Laboratory, ME). The mice were maintained under a 12-hour light/dark cycle with free access to food and water. Both male and female littermates aged 3 and 6 months were used for the experiments. For histological analysis, the mice were anesthetized with carbon dioxide and perfused intracardially with 0.9% normal saline. After fixation by 4% paraformaldehyde (PFA) in 0.1 M PBS, brains were removed, placed in the same fixative solution at 4° C. overnight, and then transferred to 30% sucrose. Cryoprotected brains were serially sectioned at 40 μm in the coronal plane using a cryostat (CM1950; Leica, Wetzlar, Germany) and stored at 4° C. in Dulbecco's phosphate-buffered saline (DPBS) solution containing 0.1% sodium azide. For biochemical and proteomic analysis, the mice were anesthetized with carbon dioxide, intracardially perfused with 0.9% normal saline. Brains were then harvested, rinsed with ice-cold PBS, dissected to isolate the cortex and hippocampus for analysis. The isolated tissues immediately snap-frozen, and stored at −80° C. Blood was extracted before cardiac perfusion, and approximately 500 μl of whole blood was transferred to EDTA-coated container (BD, NJ, USA) and centrifuged at 3000 rpm, 4° C. for 15 minutes to obtain plasma.

### Immunohistochemistry

Brain sections prepared from the mice were blocked in Tris-buffered saline/0.1% Triton X-100/3% goat serum (TBS-TS) for 30 min, and incubated with a primary antibody (anti-6E10; mouse monoclonal; BioLegend, San Diego, CA) in TBS-TS overnight at 4° C. They were washed in TBS, then incubated with a secondary anti-mouse IgG labeled with Alexa Fluor 568 for 3 hrs at room temperature, washed with TBS again, and mounted onto slides using VECTASHIELD® Antifade Mounting Medium with DAPI (Vector Laboratories, Newark, CA). Images were acquired using Pannoramic scan system (3DHistech, Budapest, Hungary).

Peptide Generation Via in-Solution Brain Tissue Digestion

Hippocampal and cortical tissues were dissected from 3- and 6-month-old 5xFAD mouse brains and rinsed with PBS. Each tissue sample was lysed in a lysis buffer (40 mM ammonium bicarbonate (ABC), pH 7.8) supplemented with 1% proteaseMAX (Promega, Madison, WI, USA). After ultrasonication and incubation on ice for 30 min, the lysates were diluted fourfold with 40 mM ABC. The sample was incubated with 10 mM dithiothreitol for 20 minutes at 56° C. and then treated with 20 mM iodoacetamide for 20 minutes at room temperature in the dark. Protein concentrations were quantified using a BCA protein assay kit, and 100 μg of protein was digested with a 1:50 trypsin-Lys C mixture (Promega Madison, WI, USA) at 50° C. for 4 hours. The digestion reaction was terminated with 0.5% trifluoroacetic acid (TFA), and the trypsin-digested peptides were dried using a lyophilizer. Desalting was performed using desalting columns (#89873, Thermo Fisher Scientific) according to the manufacturer's protocol to obtain peptides digested with trypsin from the brain tissues.

Isolation of Extracellular Vesicles from Blood

Plasma was obtained from the collected whole blood by treatment with EDTA and centrifugation. The plasma was diluted 10-fold with PBS and incubated at 4° C. for 60 minutes. The diluted solution was centrifuged at 12,000 rpm for 20 minutes at 4° C. The pellet thus formed was resuspended in 1 ml PBS and centrifuged twice at 120,000×g for 90 minutes at 4° C. The precipitated pellet was finally resuspended in 200 μl PBS. The EV concentration was measured using a BCA protein assay, and their size was determined using NanoSight LM10 (Malvern Instruments) following the manufacturer's instructions.

Peptide Generation from Plasma-Derived Extracellular Vesicles

EVs isolated from plasma were lysed in a lysis buffer containing 1% proteaseMAX and 40 mM ABC (pH 7.8). After ultrasonication and a 30-minute incubation on ice, the lysates were diluted fourfold with 40 mM ABC. The sample was incubated with 10 mM dithiothreitol for 20 minutes at 56° C. and then treated with 20 mM iodoacetamide for 20 minutes at room temperature in the dark. Protein concentrations were quantified using a BCA protein assay kit, and 100 μg of protein was digested with a 1:50 trypsin-Lys C mixture (Promega Madison, WI, USA) at 50° C. for 4 hours. The trypsin-digested peptides were dried using a lyophilizer. Desalting was performed using desalting columns (#89873, Thermo Fisher Scientific) according to the manufacturer's protocol to obtain peptides.

### Mass Spectrometry and Data Analysis

The trypsin-digested peptides were analysed using a liquid chromatography-tandem mass spectrometry system (LC-MS/MS) consisting of an UltiMate™ 3000 RSLCnano system (Thermo Fisher Scientific, Waltham, MA, USA) and an Orbitrap Eclipse Tribrid mass spectrometer (Thermo Fisher Scientific) equipped with a nano-electrospray source (EASY-Spray Sources, Thermo Fisher Scientific). Peptides were trapped in a 75 μm×2 cm C18 pre-column (nanoViper, Acclaim PepMap100, Thermo Fisher Scientific) before being separated on an analytical C18 column (75 μm×50 cm PepMap RSLC, Thermo Fisher Scientific). The peptides were separated with a 140-min discontinuous gradient of 5-25% acetonitrile and 0.1% formic acid at a flow rate of 250 nL/min. The voltage applied to produce an electrospray was 2000 V. During the chromatographic separation, the Orbitrap Eclipse Tribrid was operated in data-dependent mode, automatically switching between MS1 and MS2.

The mass spectrometry (MS) data were acquired using the following parameters: Full scan MS1 spectra (400-1600 m/z) were acquired for a maximum ion injection time of 100 ms at a resolution of 60,000 and an automatic gain control (AGC) target value of 4.0e5 in the Orbitrap. MS2 spectra were acquired in the Orbitrap mass analyser at a resolution of 60,000 with high energy collision dissociation (HCD) of 30% normalised collision energy and AGC target value of 1.0e5 with a maximum ion injection time of 300 ms. Previously fragmented ions were excluded for 20 seconds. Mass spectrometer calibration was performed with the proposed calibration solution according to the manufacturer's instructions.

To perform a database search, tandem mass spectra data was processed with Thermo Fisher Scientific Proteome Discoverer software version 2.41. The spectral data were searched against the mouse Uniprot database (release version 2020_09). The analysis workflow used included the four nodes, Spectrum Files (data input), Spectrum Selector (spectrum and feature retrieval), Sequest HT (sequence database search), and Percolator (peptide spectral match (PSM) or PSM validation and false discovery rate (FDR) analysis). All identified proteins had an FDR of 1%, which was calculated at the peptide level. Validation was based on q-value. Search parameters allowed for tryptic specificity of up to two missed cleavages, with methylthio-modifications of cysteine as a fixed modification, and oxidation of methionine as a dynamic modification. The mass search parameters for +1, +2, and +3 ions included mass error tolerances of 20 ppm for precursor ions and 0.6 Da for the fragment ions.

To calculate quantitative changes in the identified proteins among the experimental groups, the normalised peptide spectrum match index was applied. Calculation was made of the peptide spectrum match index of each protein, which is the cumulative peptide spectrum match from each technical replicate. To estimate statistical confidence for fold changes of identified proteins between experimental groups, the G-test for peptide spectrum matches was used.

### Bioinformatics Analysis

DAVID Bioinformatics Resources 6.8 was used for gene ontology (GO)-based function annotation, while IPA was utilized for in-depth bioinformatics analysis. For the identified proteins, Uniprot protein accession numbers coupled with the value of the normalised fold changes between WT and 5xFAD were uploaded to IPA in the protein expression criteria. For quantitative pathway analysis, the z-score cutoff was set at 0.5, and −log(p-value)>1.3 was applied.

### Western Blot

Western blotting for protein analysis was performed as follows. Total proteins were extracted using a radioimmunoprecipitation assay (RIPA) buffer containing 1× Halt protease and phosphatase inhibitor cocktail (Thermo Fisher Scientific, USA). Measurement of protein concentration was calculated by bicinchoninic acid (BCA) protein assay (Thermo Fisher Scientific). Protein samples were mixed with sodium dodecyl-sulphate (SDS) sample buffer (Bio-Rad) containing 10% beta-mercaptoethanol, and then boiled for 5 minutes. After protein separation by SDS-polyacrylamide gel electrophoresis, the proteins were transferred to polyvinylidene difluoride membranes (Millipore, USA) using the Bio-Rad wet transfer system, blocked by TBS-T containing 5% skimmed milk for 30 min, and incubated with primary antibodies overnight at 4° C. Thereafter, the membranes were washed three times with TBS-T, and the blots were incubated with anti-mouse or anti-rabbit IgG horseradish peroxidase (HRP)-conjugated secondary antibodies (GeneTex, USA) for 1 hour at room temperature (±25° C.). The membranes were then washed with TBS-T and developed using enhanced chemiluminescence (ECL) solution. The antibodies used in the Western blot are listed in Table 1.

TABLE 1

| | | | Dilution | |
|---|---|---|---|---|
| Target name | Cat no. | Host | titer | Company |
| ALG-2-interacting | ab275377 | Rabbit | 1:500 | Abcam |
| protein X (ALIX) | | | | |
| Alpha-2-macroglobin | ab58703 | Rabbit | 1:500 | Abcam |
| (A2M) | | | | |
| Phospho protein kinase | 9271S | Rabbit | 1:1,000 | Cell Signaling |
| B (Akt) | | | | Technology |
| Total Akt | 9272S | Rabbit | 1:1,000 | Cell Signaling |
| | | | | Technology |
| Amyloid precursor | MAB348 | Mouse | 1:1,000 | Sigma-Aldrich |
| protein (APP) | | | | |
| Cluster of differentiation | sc-5275 | Mouse | 1:1,000 | Santa Cruz |
| 63 (CD63) | | | | Biotechnology |
| CD9 | MA5-31980 | Rabbit | 1:1,000 | Invitrogen |
| Creatine kinase M-type | ab233201 | Rabbit | 1:500 | Abcam |
| (CKM) | | | | |
| Filamin-A (FLNA) | LS-B7557-50 | Rabbit | 1:500 | LS Bio |
| FLNC | ARP64454_P050 | Rabbit | 1:500 | AVIVA System |
| Haptoglobin (Hp) | ab256454 | Rabbit | 1:10,000 | Abcam |
| Heat shock protein 70 | MA3-006 | Mouse | 1:500 | Invitrogen |
| (HSP70) | | | | |
| Integrin alpha-IIb | MBS2521429 | Rabbit | 1:500 | MyBioSource |
| (ITGA2B) | | | | |
| Lysosomal alpha- | MBS9130534 | Rabbit | 1:500 | MyBioSource |
| mannosidase (MAN2B1) | | | | |
| Orosomucoid 2 (ORM2) | ab231906 | Rabbit | 1:500 | Abcam |
| Platelet factor 4 (PF4) | ab129183 | Rabbit | 1:500 | Abcam |
| phospholipid transfer | LS-B295 | Rabbit | 1:500 | LS Bio |
| protein (PLTP) | | | | |
| isoform 3 of quiescin | 12713-1-AP | Rabbit | 1:500 | Proteintech |
| sulfhydryl oxidase 1 | | | | |
| (QSOX1) | | | | |
| protein-glutamine | PA5-29357 | Rabbit | 1:500 | Invitrogen |
| gamma- | | | | |
| glutamyltransferase 2 | | | | |
| (TGM2) | | | | |
| Talin 1 (TLN1) | ab108480 | Mouse | 1:500 | Abcam |
| β-actin | Sc-47778 | Mouse | 1:1000 | Santa Cruz |
| | | | | Biotechnology |

### Preparation of Alzheimer's Disease Patient-Derived Specimen

Human blood plasma samples (n=125) were acquired from normal group (healthy individuals) and patients with early-stage AD and late-stage AD at the Chungbuk National University Hospital Biobank (Cheongju, Korea) and the Korea Biobank Network (Yongin, Korea). The blood plasma was used to prepare plasma EVs as described in the foregoing and processed according to manufacturer's instructions. Briefly, 200 μL of plasma was incubated with 50.4 μL of ExoQuick exosome precipitation solution (System Biosciences, CA, USA) for 30 minutes at 4° C. After incubation, the Exoquick/plasma mixture was centrifuged at 1,500×g for 30 minutes at 4° C., and the EV pellet thus formed was resuspended in 50 μL of 1× Dulbecco's Phosphate Buffered Saline (1×DPBS, Hyclone, USA). The isolated EVs were used as a sample to obtain proteins. All participants signed an informed consent form, and all protocols were approved by the institutional review board of the National Biobank of Korea. All the groups comprised 54- to 90-year-old individuals without distinction of sex. The groups of patients with early- and late-stage AD were defined by mini-mental state examination (MMSE) scoring (Late<16, 16 Early<23, 24 Normal). All plasma samples were collected by fine-needle aspiration and pre-operative blood draw with heparin and were stored at −80° C. until use.

### Machine Learning

To assess the performance of the proteins suggested as the Alzheimer's disease biomarkers, a support vector machine (SVM), which is a classifier used for the separation of the data sets, was employed. Three classification models for normal vs. early Alzheimer's disease, early Alzheimer's disease vs. late Alzheimer's disease, and normal vs. late Alzheimer's disease were constructed and validated because conventional SVM is designed for binary classification. All of features should be required to include that protein markers had to be fully registered in each class (normal, early Alzheimer's disease, and late Alzheimer's disease). Selected features were accumulated via t-test based scoring. Finally, nine proteins were included by common intersection among all of classes. The classification accuracies were evaluated using 10x10-fold cross-validations. Classification performance was also tested using area under curve and receiver operating characteristic curve (AUC-ROC). All machine learning processes were carried out using MATLAB R2019b (Mathworks, Inc., Natick, MA, USA).

### Statistical Analysis

All data were expressed as mean±SEM. Statistical significance of differences between groups was determined using two-tailed t-tests or one-way analysis of variance (ANOVA) followed by Bonferroni's multiple comparison test, performed with Prism version 9.0 (GraphPad Software Inc., CA). p values of less than 0.03 was considered statistically significant.

### Example 1

### Proteomics Analysis of Plasma-Derived Extracellular Vesicles in Normal and Alzheimer's Model Mice

<1-1> Verification of Extracellular Vesicles Isolated from Plasma

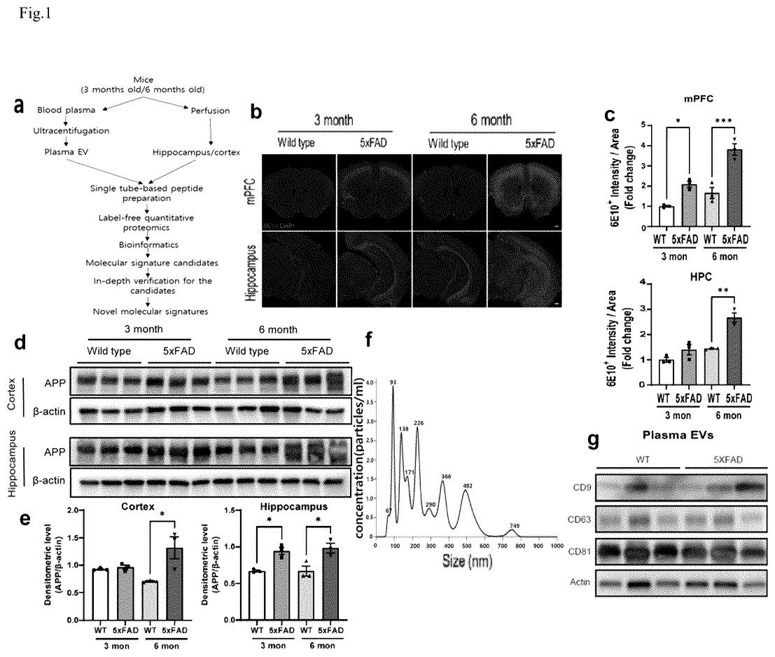

To explore novel molecular signature for Alzheimer's disease, the workflow illustrated in FIG. 1a was conducted. First, the level of β-amyloid (AB) was checked to determine whether the 5xFAD mice used in the experiments exhibited Alzheimer's disease. The results revealed significant accumulation of AB plaques in the medial prefrontal cortex (mPFC) and hippocampus of 3-month-old 5xFAD mice, with a greater degree of accumulation observed in 6-month-old 5xFAD mice compared to 3-month-old mice (FIG. 1b). In contrast, wild-type (WT) mice displayed slight accumulation of AB plaques in the CA1 and subiculum of hippocampus at 6 months of age, but with no significance, and there were no significant differences observed in the mPFC (FIGS. 1b and 1c). Furthermore, levels of amyloid precursor protein (APP) were significantly elevated in the hippocampus of 3-month-old 5xFAD mice compared to WT mice, and this increase was significant in both the cortex and hippocampus of 6-month-old 5xFAD mice (FIGS. 1d and 1e). These findings confirmed that the 5xFAD mice utilized in this experiment exhibited key characteristics of Alzheimer's disease.

Next, to detect AD-dependent altered proteins in plasma EVs, plasma was separated from the blood of Alzheimer's model 5xFAD and WT mice and ultracentrifuged to obtain plasma EVs. The quality and purity of the isolated EVs were assessed by size measurement using NanoSight LM10 (Malvern PANalytical, Malvern, UK). Most isolated EVs were measured to be 100 nm in length (FIG. if). Additionally, the key EV biomarker proteins, including Cluster of Differentiation (CD)9, CD63, and CD81, were verified to be abundant (FIG. 1g).

These results demonstrate that plasma-derived EVs were successfully isolated from both normal and Alzheimer's model mice.

### <1-2> Proteomics Analysis of Multi-Proteomes in Normal and Alzheimer's Model Mice

Proteomics analyses were conducted on the cortex, hippocampus, and plasma-derived EVs isolated from both normal and Alzheimer's model mice. In addition, the mice included 3-month-old and 6-month-old mice.

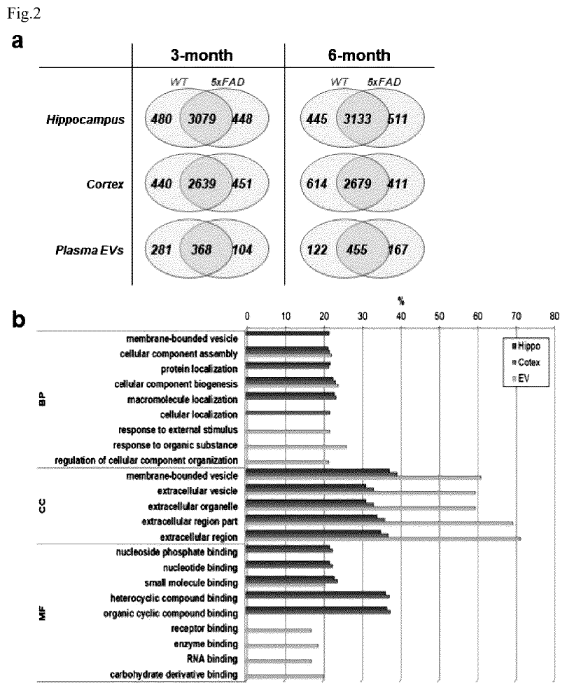

In 3-month-old normal and Alzheimer's model mice, proteomic analysis identified 4,007, 3,530, and 753 proteins in the hippocampus, cortex, and plasma-derived EVs, respectively. Similarly, in 6-month-old normal and Alzheimer's model mice, 4,089, 3,704, and 744 proteins were identified in the hippocampus, cortex, and plasma-derived EVs, respectively (FIG. 2a). Bioinformatics analysis was applied to the proteomic data from 3-month-old mice. In enrichment of functional annotation, the hippocampus and cortex proteomes shared gene ontology (GO) terms under biological processes (BP), cellular components (CC), and molecular functions (MF) (FIG. 2b). Under GO-BP, the hippocampus and cortex proteomes shared the same cellular component-related terms and protein localisation except for membrane-bounded vesicle and cellular localisation. Under GO-CC, five major terms including extracellular vesicle, organelle, and their related terms were common in both the hippocampus and cortex proteomes. Under GO-MF, the hippocampus and cortex proteomes shared five major terms including nucleoside phosphate, nucleotide, small molecule, heterocyclic compound, and organic cyclic compound binding. However, the proteome of the plasma EV distinctly differed from those of the hippocampus and cortex with respect to their GO terms and percent involvement. Under GO-BP, the plasma EV proteome included unique GO terms such as response to external stimulus, organic substance, and regulation of cellular component organisation. Under GO-CC, the proteome of the plasma EV shared the same GO terms with those of the hippocampus and cortex. Nevertheless, the percent involvement of these terms was relatively higher in the proteome of the plasma EV than in those of the hippocampus and cortex. Under GO-MF, the terms differed between the plasma EV and the other proteomes.

### <1-3> Proteomics Analysis of Multi-Proteomes in 3-Month-Old and 6-Month-Old Alzheimer's Model Mice

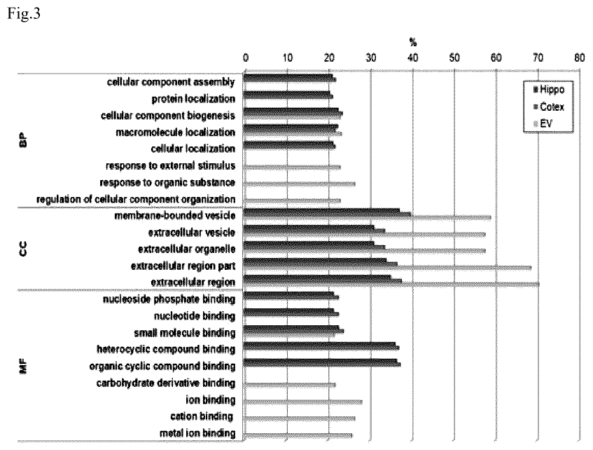

Further proteomics analyses were conducted on hippocampus, cortex, and plasma-derived EVs from 3-month-old and 6-month-old Alzheimer's model mice, and the results are depicted in FIG. 3.

As shown in FIG. 3, the analysis results indicated that the proteomes derived from 3-month-old and 6-month-old Alzheimer's model mice shared GO terms under biological process (BP), cellular component (CC), and molecular function (MF). Under GO-BP, the proteomes in the 3-month-old and 6-month-old Alzheimer's model mice shared the same cellular component-related terms and protein and macromolecule localisation while under GO-CC and GO-MF, the proteomes from 3-month-old mice were summarized as those from the hippocampus and cortex of 6-month-old mice. The plasma EV proteomes were distinct with respect to the categories and percent involvement of their GO terms. The 3-month-old and 6-month-old Alzheimer's model plasma EV proteomes included unique GO terms (FIG. 3). Particularly under GO-MF, there were dramatic differences between the plasma EV proteomes and the hippocampus and cortex proteomes.

### Example 2

Selection of Biomarker Candidates for Alzheimer's Disease from Plasma-Derived Extracellular Vesicles

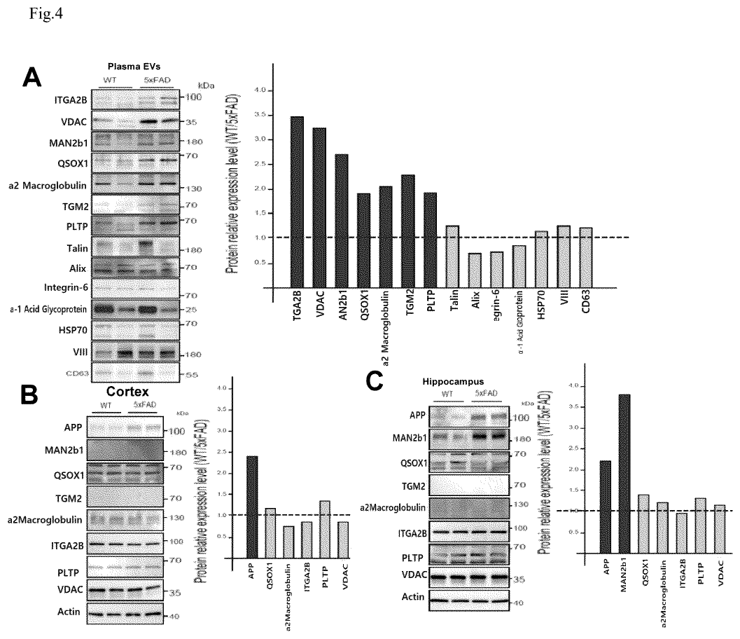

Through quantitative analysis of multi-proteomes, plasma EV proteins that exhibited increased expression in Alzheimer's model mice compared to the control group were identified and listed in Table 2. The candidate proteins identified from the cortex, hippocampus, and plasma-derived EVs of 3-month-old Alzheimer's model mice were further analyzed via Western blot and the results are depicted in FIG. 4. In Table 2, “EV” refers to extracellular vesicles, “Ctx” to the cortex, and “Hippo” to the hippocampus.

TABLE 2

| | | | 3-month-old | | 6-month-old | | 3-month-old | |
|---|---|---|---|---|---|---|---|---|
| | | | EV | | EV | | Hippo | |
| # | Description | Gene ID | Log₂Fold | P | Log₂Fold | P | Log₂Fold | P |
| 1 | Integrin alpha-IIb | Itga2b | −1.3 | 0.0001 | 0.6 | 0.0253 | −0.6 | 0.6532 |
| 2 | Talin-1 | Tln1 | −1.3 | 0.0000 | 0.6 | 0.0108 | 0.7 | 0.1953 |
| 3 | Filamin-A | Flna | −2.0 | 0.0000 | 0.5 | 0.0174 | ND | ND |
| 4 | Integrin alpha-6 | Itga6 | −2.7 | 0.0005 | 1.1 | 0.0304 | ND | ND |
| 5 | Protein-glutamine gamma- | Tgm2 | −3.8 | 0.0000 | 4.3 | 0.0042 | −0.6 | 0.6532 |
| | glutamyltransferase 2 | | | | | | | |
| 6 | Major urinary protein 2 | Mup2 | 6.3 | 0.0000 | 4.0 | 0.0109 | ND | ND |
| 7 | Alpha-1-acid glycoprotein 2 | Orm2 | 4.3 | 0.0029 | 2.8 | 0.0335 | ND | ND |
| 8 | Murinoglobulin-2 | Mug2 | ND | ND | 6.7 | 0.0000 | ND | ND |
| 9 | Filamin-C | Flnc | ND | ND | 4.6 | 0.0010 | −1.0 | 0.6823 |
| 10 | ATP-dependent 6-Phosphofructokinase, | Pfkm | ND | ND | 4.6 | 0.0010 | 0.0 | 0.8533 |
| | muscle type | | | | | | | |
| 11 | Isoform 2 of Alpha-crystallin A chain | Cryaa | ND | ND | 4.4 | 0.0026 | ND | ND |
| 12 | Heat shock 70 kDa protein 1-like | Hspa1l | ND | ND | 4.0 | 0.0109 | 0.0 | 0.9405 |
| 13 | Voltage-dependent anion-selective | Vdac1 | ND | ND | 4.0 | 0.0109 | 0.1 | 0.4452 |
| | channel protein 1 | | | | | | | |
| 14 | Creatine kinase M-type | Ckm | ND | ND | 3.8 | 0.0177 | −0.1 | 0.8240 |
| 15 | Alpha-2-macroglobulin-P | A2m | 5.0 | 0.0001 | ND | ND | ND | ND |
| 16 | Isoform 3 of Sulfhydryl oxidase 1 | Qsox1 | 4.3 | 0.0029 | ND | ND | ND | ND |
| 17 | Haptoglobin | Hp | 7.1 | 0.0000 | ND | ND | 1.0 | 0.6839 |
| 18 | H-2 class I histocompatibility | H2-L | 4.3 | 0.0029 | ND | ND | ND | ND |
| | antigen, L-D alpha chain | | | | | | | |
| 19 | Phospholipid transfer protein | Pltp | 3.5 | 0.0036 | ND | ND | ND | ND |
| 20 | Lysosomal alpha-mannosidase | Man2b1 | 3.3 | 0.0057 | ND | ND | ND | ND |
| 21 | Platelet factor 4 | Pf4 | 2.4 | 0.0000 | −3.2 | 0.0000 | ND | ND |

| | | 6-month-old | | 3-month-old | | 6-month-old | | |
|---|---|---|---|---|---|---|---|---|
| | | Hippo | | Ctx | | Ctx | | Clin. |
| # | Description | Log₂Fold | P | Log₂Fold | P | Log₂Fold | P | trial |
| 1 | Integrin alpha-IIb | −2.6 | 0.1829 | −1.0 | 0.6799 | 0.2 | 0.9273 | X |
| 2 | Talin-1 | 1.0 | 0.0296 | 0.2 | 0.6959 | 0.2 | 0.7239 | X |
| 3 | Filamin-A | 0.7 | 0.2041 | −3.0 | 0.0191 | 3.4 | 0.0062 | X |
| 4 | Integrin alpha-6 | ND | ND | ND | ND | ND | ND | X |
| 5 | Protein-glutamine gamma- | 0.5 | 0.5584 | 0.0 | 0.9960 | 0.2 | 0.8745 | X |
| | glutamyltransferase 2 | | | | | | | |
| 6 | Major urinary protein 2 | ND | ND | ND | ND | ND | ND | X |
| 7 | Alpha-1-acid glycoprotein 2 | ND | ND | ND | ND | ND | ND | X |
| 8 | Murinoglobulin-2 | ND | ND | ND | ND | ND | ND | X |
| 9 | Filamin-C | ND | ND | ND | ND | ND | ND | X |
| 10 | ATP-dependent 6-Phosphofructokinase, | 0.1 | 0.6199 | 0.0 | 0.7990 | 0.0 | 0.9104 | X |
| | muscle type | | | | | | | |
| 11 | Isoform 2 of Alpha-crystallin A chain | ND | ND | ND | ND | ND | ND | X |
| 12 | Heat shock 70 kDa protein 1-like | 0.2 | 0.3304 | 0.0 | 0.9587 | −0.1 | 0.7915 | X |
| 13 | Voltage-dependent anion-selective | −0.1 | 0.5607 | 0.0 | 0.8182 | −0.2 | 0.1824 | X |
| | channel protein 1 | | | | | | | |
| 14 | Creatine kinase M-type | 0.4 | 0.5060 | 0.0 | 0.9887 | 0.2 | 0.8232 | X |
| 15 | Alpha-2-macroglobulin-P | ND | ND | ND | ND | ND | ND | X |
| 16 | Isoform 3 of Sulfhydryl oxidase 1 | ND | ND | ND | ND | ND | ND | X |
| 17 | Haptoglobin | −2.6 | 0.0596 | 1.0 | 0.6863 | −3.1 | 0.0765 | O |
| 18 | H-2 class I histocompatibility | ND | ND | ND | ND | ND | ND | X |
| | antigen, L-D alpha chain | | | | | | | |
| 19 | Phospholipid transfer protein | −1.0 | 0.6852 | ND | ND | ND | ND | X |
| 20 | Lysosomal alpha-mannosidase | ND | ND | ND | ND | ND | ND | X |
| 21 | Platelet factor 4 | ND | ND | ND | ND | ND | ND | X |

The analysis revealed that integrin alpha-IIb (ITGA2B), voltage-dependent anion-selective channel protein (VDAC), lysosomal alpha-mannosidase (MAN2B1), sulfhydryl oxidase 1 (QSOX1), alpha-2-macroglobulin (A2M), protein-glutamine gamma-glutamyltransferase 2 (TGM2), and phospholipid transfer protein (PLTP) were significantly upregulated in the plasma EVs of 3-month-old Alzheimer's model mice compared to the control group (FIG. 4a). Analysis of the candidate proteins in the cortex and hippocampus revealed that only the expression of MAN2B1 was significantly altered (FIGS. 4b and 4c). Based on these findings, the proteins with significantly altered expression levels are listed in Table 2 and were identified as potential biomarkers for the early diagnosis of Alzheimer's disease.

### Example 3

Selection of Biomarkers for Diagnosis of Early Alzheimer's Disease Using Plasma EVs from Early- and Late-Stage AD Patients

To validate the practical availability of the biomarkers of Table 2 identified in Example 2 as diagnostic biomarkers for Alzheimer's disease, plasma EVs were collected from early-stage and late-stage Alzheimer's disease patients and analyzed for the protein levels of the biomarkers. Plasma EVs from healthy individuals were used as controls.

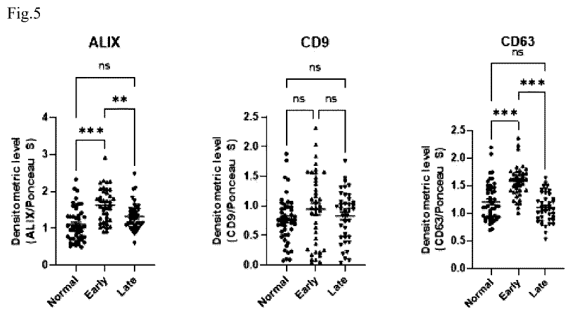

Specifically, plasma samples were obtained from patients classified as normal, early-stage Alzheimer's disease (early-stage AD), and late-stage Alzheimer's disease (late-stage AD) based on Mini-Mental State Examination (MMSE) scores. Plasma EVs were isolated, and the protein expression levels of the candidate biomarkers were analyzed via Western blot. The presence of EV markers, including alix, CD9, and CD63, confirmed the successful isolation of EVs from plasma samples of healthy individuals and AD patients (FIG. 5).

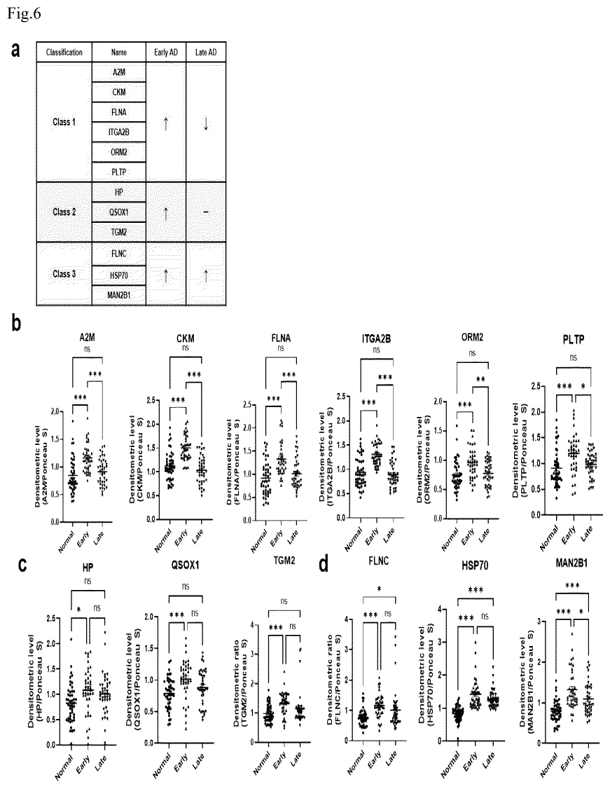

The analysis, as presented in FIG. 6, showed that the protein levels of 12 candidate biomarkers including A2M, CKM, FLNA, ITGA2B, ORM2, PLTP, HP, QSOX1, TGM2, FLNC, HSP70, and MAN2B1 were significantly upregulated in the plasma EVs of early-stage Alzheimer's disease patients compared to those of normal individuals and late-stage Alzheimer's disease patients (FIG. 6a).

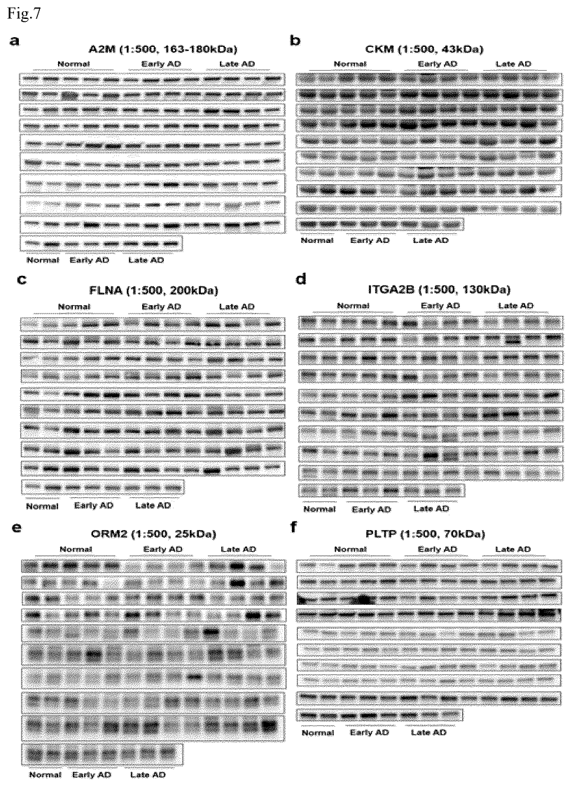

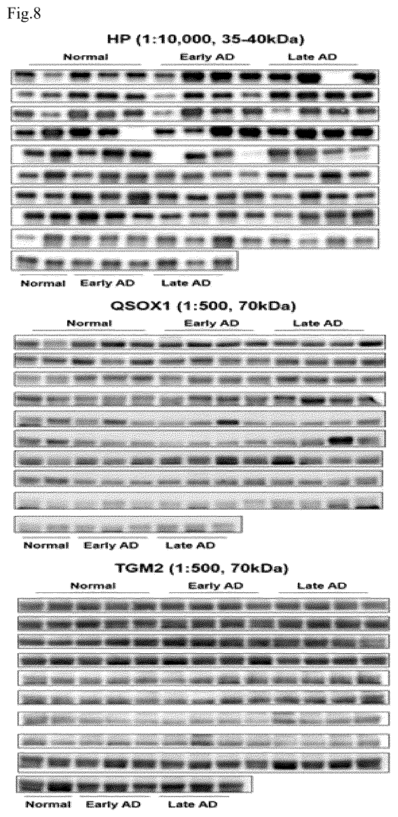

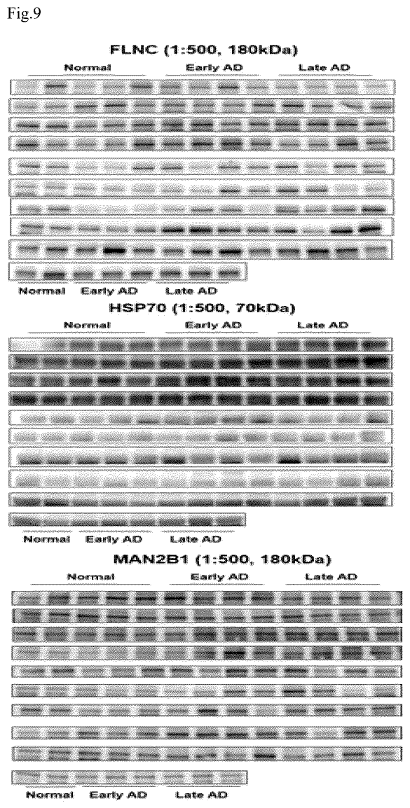

Specifically, the expression patterns of the candidate markers were sorted into three classes as follows. Class 1 group (A2M, CKM, FLNA, ITGA2B, ORM2, PLTP): These proteins were significantly upregulated only in early-stage AD patients, with no notable differences in late-stage AD patients compared to the control group (FIG. 6b); Class 2 group (HP, QSOX1, TGM2): These proteins were significantly upregulated in early-stage AD patients compared to the control group (FIG. 6c). Class 3 group (FLNC, HSP70, MAN2B1): These proteins were significantly upregulated in both early and late-stage AD patients compared to the control group (FIG. 6d). Western blot results for the individual protein levels in Classes 1, 2, and 3 are shown in FIGS. 7-9.

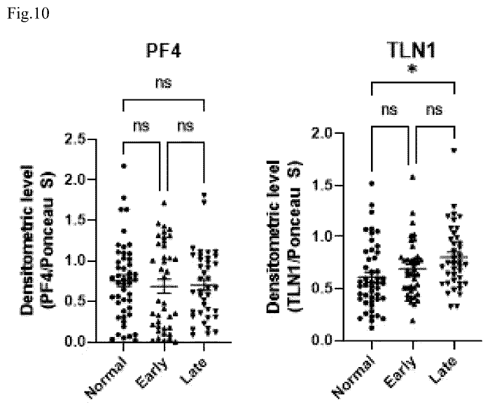

The levels of PF4 and TLN1 proteins exhibited significant individual variability and no meaningful group differences (FIG. 10).

These results obtained from the plasma EV samples of practical AD patients confirm that the biomarkers A2M, CKM, FLNA, ITGA2B, ORM2, PLTP, HP, QSOX1, TGM2, FLNC, HSP70, and MAN2B1 can serve as novel diagnostic tools for early-stage Alzheimer's disease.

### Example 4

### Machine Learning to Determine Optimal Biomarker Combinations for Diagnosis of Early Alzheimer's Disease

Machine learning analysis was performed on the biomarkers for early Alzheimer's disease diagnosis validated from AD patient samples in Example 3 to identify the most accurate, specific, and sensitive combinations of diagnostic biomarkers. Markers identified in Example 3 (ITGA2B, CKM, FLNC, MAN2B1, TGM2, A2M, FLNA, ORM2, and PLTP) were analyzed. Diagnostic accuracy, specificity, and sensitivity were assessed for various combinations of selected markers across three groups: Normal vs. early-stage AD; Normal vs. late-stage AD; and Early-stage AD vs. late-stage AD. Performance of marker combinations was evaluated using AUC-ROC curves, which represent the tradeoff between sensitivity and specificity across all tests.

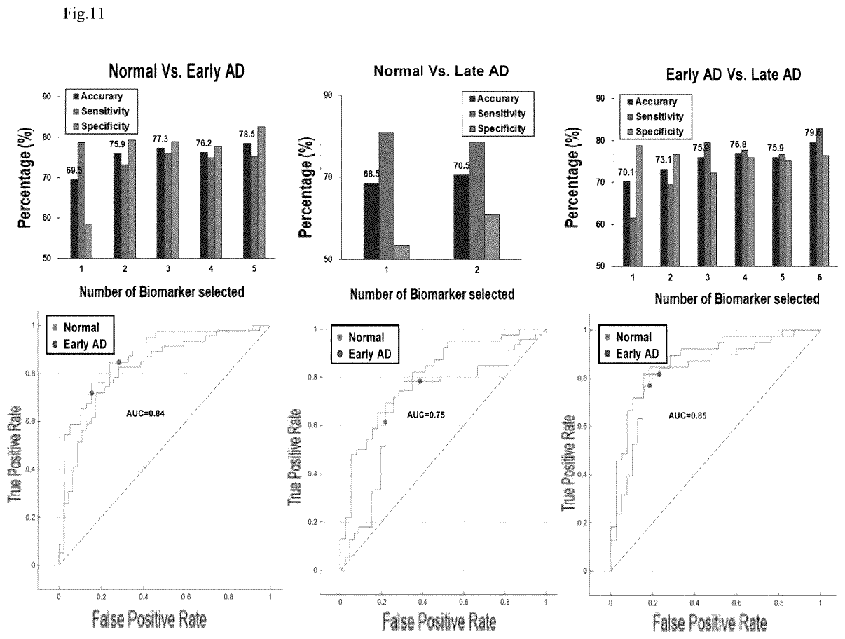

As shown in FIG. 11, in the support vector machine (SVM) classifiers of normal vs. early-stage AD, the combination of five biomarkers (ITGA2B, FLNC, CKM, TGM2, and MAN2B1) achieved the highest diagnostic accuracy (78.49%), sensitivity, and specificity for distinguishing normal individuals from early-stage AD patients. Classification performance of normal vs. early-stage AD was validated by AUC of 0.84, indicating reliable diagnostic capability.

A combination of two biomarkers (MAN2B1 and FLNC) among the markers selected in the present disclosure achieved 70.47% accuracy for distinguishing normal individuals from late-stage AD patients. A combination of six biomarkers (CKM, ITGA2B, A2M, ORM2, PLTP, and FLNA) achieved 79.62% accuracy for distinguishing early-stage AD from late-stage AD patients.

The AUCs for distinguishing normal vs. late-stage AD and early-stage vs. late-stage AD were 0.75 and 0.85, respectively. More than 0.8 has been accepted to excellent performance of the suggested classification model.

Taken together, the data obtained above demonstrate that the 12 candidate biomarkers identified in the present disclosure (A2M, CKM, FLNA, ITGA2B, ORM2, PLTP, HP, QSOX1, TGM2, FLNC, HSP70, and MAN2B1) enable highly accurate, sensitive, and specific diagnosis of early Alzheimer's disease using blood samples. The optimal combination of five biomarkers (ITGA2B, FLNC, CKM, TGM2, MAN2B1) provided the best diagnostic performance.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A biomarker composition for diagnosing early-stage Alzheimer's disease, the composition comprising at least one gene selected from the group consisting of A2M (alpha-2-macroglobulin), CKM (creatine kinase M-type), FLNA (filamin-A), ITGA2B (Integrin alpha-IIb), ORM2 (alpha-1-acid glycoprotein 2), PLTP (phospholipid transfer protein), HP (haptoglobin), QSOX1 (sulfhydryl oxidase 1), TGM2 (protein-glutamine gamma-glutamyltransferase 2), FLNC (filamin C), HSP70 (heat shock protein 70), and MAN2B1 (lysosomal alpha-mannosidase), or a protein expressed from the gene.

2. The biomarker composition of claim 1, wherein the gene or protein exhibited an elevated expression level during the onset of Alzheimer's disease compared to normal individuals.

3. The biomarker composition of claim 1, the composition comprising 4 to 6 different types of the genes or the proteins expressed from the genes.

4. A composition for early Alzheimer's disease diagnosis, the composition comprising a substance for measuring mRNA or protein level of at least one gene selected from the group consisting of A2M(alpha-2-macroglobulin), CKM(creatine kinase M-type), FLNA(filamin-A), ITGA2B(Integrin alpha-IIb), ORM2(alpha-1-acid glycoprotein 2), PLTP(phospholipid transfer protein), HP(haptoglobin), QSOX1(sulfhydryl oxidase 1), TGM2(protein-glutamine gamma-glutamyltransferase 2), FLNC(filamin C), HSP70(heat shock protein 70), and MAN2B1(lysosomal alpha-mannosidase).

5. The composition of claim 4, wherein the composition comprises a substance for measuring mRNA or protein levels of four to six different types of the genes.

6. The composition of claim 4, wherein the substance is a primer, probe or antibody that specifically binds to the gene or protein.

7. A diagnostic kit for early Alzheimer's disease, the kit comprising the composition of claim 4.

8. A method for providing information for predicting and diagnosing Alzheimer's disease, the method comprising the steps of:
(a) measuring an mRNA level or protein expression level of at least one selected from the group consisting of A2M(alpha-2-macroglobulin), CKM(creatine kinase M-type), FLNA(filamin-A), ITGA2B(Integrin alpha-IIb), ORM2(alpha-1-acid glycoprotein 2), PLTP(phospholipid transfer protein), HP(haptoglobin), QSOX1(sulfhydryl oxidase 1), TGM2(protein-glutamine gamma-glutamyltransferase 2), FLNC(filamin C), HSP70(heat shock protein 70), and MAN2B1(lysosomal alpha-mannosidase) from a biological sample isolated from a subject suspected of having Alzheimer's disease; and
(b) comparing the measurement result of step (a) with the mRNA or protein expression level of the same gene in a normal control sample.

9. The method of claim 8, wherein the biological sample is blood or plasma.

10. The method of claim 9, wherein the sample is extracellular vesicles derived from plasma.

11. The method of claim 8, further comprising a step of determining the subject to be at an early stage of Alzheimer's disease when the mRNA or protein expression level of the gene is increased compared to a normal control group.

12. The method of claim 8, wherein the mRNA levels or protein expression levels of A2M(alpha-2-macroglobulin), CKM(creatine kinase M-type), FLNA(filamin-A), ITGA2B(Integrin alpha-IIb), ORM2(alpha-1-acid glycoprotein 2) and PLTP(phospholipid transfer protein) genes increase during early-stage Alzheimer's disease compared to a normal control group and subsequently decrease during late-stage Alzheimer's disease.

13. The method of claim 8, wherein the mRNA levels or protein expression levels of HP(haptoglobin), QSOX1(sulfhydryl oxidase 1), and TGM2(protein-glutamine gamma-glutamyltransferase 2) genes increase only during early-stage Alzheimer's disease.

14. The method of claim 8, wherein the mRNA levels or protein expression levels of FLNC(filamin C), HSP70(heat shock protein 70), and MAN2B1(lysosomal alpha-mannosidase) genes increase during both early and late stages of Alzheimer's disease.
